(19) United States
(12) Patent Application Publication
(10) Pub. No.: US 2023/0272023 A1
(43) Pub. Date: Aug. 31, 2023

(54) NGF VARIANTS, PRODUCTION, COMPOSITIONS, AND THERAPEUTIC USES

(71) Applicant: HUMAN CELL CO., Naperville, IL (US)

(72) Inventors: Ridong CHEN, Naperville, IL (US); Soon Seog JEONG, Naperville, IL (US)

(73) Assignee: HUMAN CELL CO., Naperville, IL (US)

(21) Appl. No.: 18/102,214

(22) Filed: Jan. 27, 2023

Related U.S. Application Data
(63) Continuation-in-part of application No. PCT/US2021/043316, filed on Jul. 27, 2021.
(60) Provisional application No. 63/056,838, filed on Jul. 27, 2020.

(51) Int. Cl. C07K 14/48

(52) U.S. Cl. CPC C07K 14/48; A61K 38/00

(57) ABSTRACT

This invention provides new NGF variants, pharmaceutically acceptable compositions thereof, methods of their production, and methods of their use to treat individuals in need of neuroprotection or stimulation of epithelial-derived cells with no pain or only tolerable pain.

### CROSS-REFERENCE TO RELATED APPLICATIONS

This application is a continuation-in-part of International Application No PCT/US21/43316, filed 27 Jul. 2021, which claims the benefit of U.S. provisional patent application Ser. No: 63/056,838 filed on 27 Jul. 2020, each of which is incorporated herein by reference in its entirety.

### SEQUENCE LISTING

This application contains a Sequence Listing XML submitted under the provisions of 37 CFR 1.831(a) and herein incorporated by reference. The Sequence Listing XML includes, in XML format, the following file:

| | Creation | Size in |
|---|---|---|
| File Name | Date | bytes |
| RC371cCONT_PCT27JUL2021.xml | May 22, 2023 | 4,096 |

### TECHNICAL FIELD

The invention is in the field of human nerve growth factor (NGF), methods of producing a biologically active NGF, and therapeutic treatment of disorders of the central nervous system, peripheral nervous system, skin, and other pathologies such as ophthalmologic diseases.

### BACKGROUND

NGF is a neurotrophic factor playing a crucial role in the neurite outgrowth and cell survival of neurons (sensory and sympathetic) (Levi-Montalcini, R., Science 237 (1987) 1154; Thoenen, H., et al., Physiol. Rev. 60 (1980) 1284; Yankner, B. A., et al., Annu. Rev. Biochem. 51 (1982) 845). NGF belongs to a cysteine-knot superfamily of growth factors assuming stable dimeric protein structure. NGF promotes the growth, differentiation and vitality of cholinergic neurons of the central nervous system (Hefti, F. J., J. Neurobiol. 25 (1994) 1418). In addition, NGF promotes regeneration of injured cells, including neurons and epithelial-derived cells (Chen J., et al. Biomed Res Intern. ID547187 (2014).

NGF binds to tyrosine kinase receptors TrkA (Neurotrophin receptor) and p75^{NTR} (a member of the TNF receptor superfamily). The binding of NGF to TrkA promotes neuron survival and proliferation while NGF binding to P75^{NTR} leads to inflammation and apoptosis (Canu, N., et al. Int J Mol Sci. 18 (2017) 1319).

Previously, mutations have been introduced to reduce the binding to p75^{NTR}: T29A, D30N, I31A, I31M, I31V, K32A, K34A, V36L, K32A/K34A/E35A (Ibanez C., et al., Cell 69 (1992) 329); Ryden, M., et al, JBC. 272 (1997) 16322). The mutant K34A retained the biological activity but with reduced protein expression compared to the wildtype. Coincident mutations of three residues, i.e. K32A, K34A, and E35A, successfully abrogated interaction with p75^{NTR}. However, protein expression level and/or the TrkA receptor stimulating activity of these muteins were reduced.

The K32R and A98I variants displayed a marked increase in p75^{NTR} receptor binding compared with the wildtype NGF, and both had also a higher binding to TrkA. However, these variants did not alter their biological activity with TrkA (Cerleton L. et al., Biochem Biophys Res Comm. 495 (2018) 700).

Another drawback of NGF treatment is that wildtype NGF mediates pain transmission and perception via TrkA-activated Erk and PLC-1γ pathways and p75^{NTR}-mediated c-jun activation of sensory nerve cells, thereby reducing or eliminating possible therapeutic applications of NGF, due in part to dose-limiting effects and poor patient compliance. Inspired by the human genetic disease “hereditary sensory and autonomic neuropathy type V (HSAN5; R100W mutation),” NGF variants harboring the mutation R100E or R100E/P61S were generated to reduce the pro-nociceptive activity of NGF (Einarsdottir E. et al, Hum Mol Genet. 15 (2004)799; Capsoni S. et al, PLoS ONE. 7(2012) e37555). Reportedly, the mutations were as effective as wildtype NGF in activating the Akt-mediated survival pathway, but reduced p75^{NTR} binding and signaling, and selectively impaired the activation of Erk and PLC-1γ pathways. However, both maximal activity and stability of the muteints were reduced (Malerba F. et al, PLoS ONE. 10 (2015)e0136425).

In humans, naturally occurring mature wildtype NGF is a 120 amino acid protein (SEQ ID NO: 2), and is the result of post-translational processing of a preproprotein consisting of 241 amino acids (SEQ ID NO. 1). Specifically, the signal peptide (prepeptide) of 18 amino acids is cleaved during translocation into the endoplasmic reticulum (ER). The resulting proprotein (proNGF) is processed at its N-terminus by removing the pro-sequence by protease cleavage. Mature human NGF shows a high degree of identity (about 90%) to rodent (murine and rat) NGF (FIG. 1). For clinical studies or therapeutic uses, NGF has to be provided in high concentrations. Submaxillary glands of mice are a natural source of NGF. However, these NGF preparations are heterogeneous mixtures of different dimers and thus not suitable for therapeutic uses. Furthermore, it is desirable to administer the human form of the protein to patients. In human tissue, however, neurotrophic factors are present only in very low concentrations.

The prosequence is a domain separate from the mature protein. These two domains are separated by an exposed protease cleavage site with a basic amino acid target sequence of the type Arg-Ser-Lys-Arg located at positions 101 to 104 of the human proNGF sequence. This motif is naturally a cleavage site for the serine endoprotease Furin or related pro-protein convertases (Seidah N. et al. Biochem, 314(1996)951).

Methods for the preparation of biologically active NGF from its inactive proform are well-known in the field of the art. For example, EP 0 994 188 B1 describes a method for the preparation of biologically active NGF from its inactive pro-form. According to this method, NGF is obtainable from recombinant insoluble inactive proNGF which solubilized in a denaturing solution. Afterwards, the solubilized proNGF is transferred into a non- or weakly denaturing solution. The denatured proNGF assumes a biologically active conformation as determined by the disulfide bonds present in native NGF. Subsequently, the prosequence of proNGF is cleaved off whereby active NGF is obtained.

Therefore, an alternative protease, Trypsin (EC 3.4.21.4), was chosen to cleave proNGF to result in a mature NGF protein. The serine protease Trypsin cleaves peptide chains at the carboxyl side of basic amino acids Arginine or Lysine.

Cleavage of the wild-type pro-NGF with Trypsin to produce NGF has shown low efficiency that obliges use of high amounts of the enzyme in order to obtain a sufficient yield of cleaved NGF. This has several drawbacks that impact the subsequent process of purification. First of all, it further decreases the selectivity of the cleavage which leads to several products of digestion. Secondly, the purification of NGF from the enzyme is necessary since the enzyme has to be absent in the final sample of the protein. This uses several purification procedures to remove the abundant Trypsin. Thus, the use of Trypsin as cleavage enzyme in the procedure of the prior art leads either to low yields of NGF or to problems of purification of the protein and inhomogeneous NGF products. Thus obtaining high quality, high efficiency, and high yield NGF remains problematic.

### SUMMARY OF THE INVENTION

The invention is drawn to a genus of NGF-like proteins (herein designated as an “NGF mutein”) with high sequence identity to human NGF (SEQ ID NO: 2) and an acidic amino acid residue at amino acid residue 34, where residue numbers refer to mature human NGF of SEQ ID NO. 2. Species of this claimed genus have reduced p75^{NTR} binding and signaling yet unexpectedly enhanced TrkA signaling and improved in vivo neuroprotection. The NGF muteins can optionally include a second acidic amino acid residue at amino acid residue 32 which can further reduce or eliminate the binding affinity to p75^{NTR}, also retain the TrkA signaling and improved in vivo neuroprotection, and optionally exhibit reduced pain, relative to wildtype NGF, upon administration to a subject. Even further surprising is that species of the invention can be provided which retain or increase themostability relative to wildtype NGF and provide high yield in expression systems.

The invention also provides compositions, especially therapeutic, pharmaceutically acceptable compositions, comprising one or more of the instant NGF-muteins.

The invention also provides a method of preparing an instant NGF mutein with high biological activity and a homogenous N-terminus by culturing cells expressing a gene encoding the NGF mutein, for example, human cells such as suspension-adapted human cells. The cells can be transformed with the gene encoding the NGF mutein and N-terminus.

The invention also provides a method of administering one or more instant NGF muteins to a subject to provide therapy, e.g. to treat a disease. Optionally, such a method provides higher efficacy and optionally reduced pain relative to the wildtype NGF.

The invention also provides bacterial, yeast, or mammalian cells transformed with a gene that produces an instant NGF mutein.

The invention also provides a plasmid carrying a gene that codes for an instant NGF mutein.

### BRIEF DESCRIPTION OF THE DRAWINGS

FIG. 1. Shows an alignment of human and mouse NGF proteins sharing 90% sequence identity.

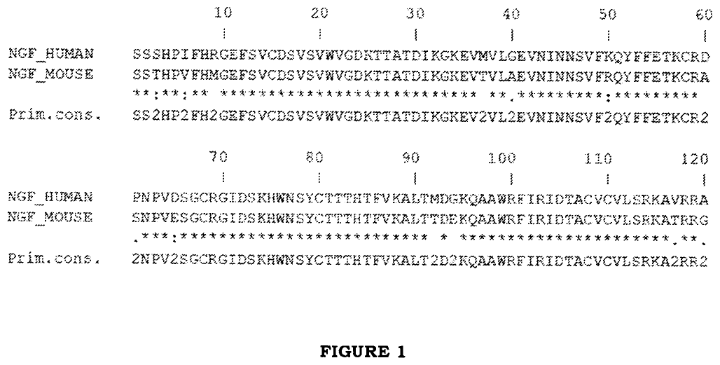

FIG. 2 shows a structure of a genus of the instant NGF muteins, where one or both Z amino acids are an acidic amino acid. It is noted that “Z”, as used herein, is not used herein as a 1 letter abbreviation for Glx (noting that Glx is known in the art to mean “glutamine or glutamic acid”). In other words, although Z can optionally be glutamic acid, Z used herein does not have the explicit definition of “glutamine or glutamic acid”.

FIG. 3 shows the nucleotide amino acid sequence of a linearized expression plasmid comprising a mouse Ig kappa signal peptide sequence and human NGF used in the Examples herein.

FIG. 4 shows the efficient production of mature NGF protein in the Expi293™ medium, while Pro-NGF is less processed in the medium of CD293 or Ex-Cell® 293.

FIG. 5 shows the expression, purification, and ex vivo activity of NGF. A. recombinant human NGF in supernatant from HEK293 cells cultured in Expi293TM medium; B. Purified NGF under non-reducing (−) or reducing (+) condition; C. Comparable cell proliferation activity with NGF vs native murine NGF. The activity was determined by the dose-dependent stimulation of the proliferation of human TF-1 cells. D. Induced outgrowth of neurite from E9 chick sympathetic ganglia with human NGF (1 ng/ml ).

FIG. 6 shows the expression of NGF muteins in Expi293™ medium. The expression level of single mutants K34A, R69A, R69E, R100D, double, and triple mutants were reduced compared to the wildtype.

FIG. 7 shows that NGF K34D stimulates the proliferation of human TF-1 cells with 3-5 fold higher potency than the wildtype without reducing thermostability.

FIG. 8 shows the experimental design of the glaucoma model study in rats (n=11-12/group).

FIG. 9 shows the animals treated with NGF K34D are healthier with better body weight gain.

FIG. 10 shows that NGF K34D effectively protects retinal ganglion cells from damage induced by elevated intraocular pressure while the wildtype or other variants have no effect or are only marginally effective.

FIG. 11 shows that NGF K34D effectively mitigates optic nerve axon damage induced by elevated intraocular pressure while the wildtype or other variants have no effect or are only marginally effective.

FIG. 12 shows the experimental design of the NGF-induced pain model study in rats.

FIG. 13 shows that the wildtype and NGF K34D induce pain with comparable potency (n=10/group).

FIG. 14 shows the expression of NGF muteins in Expi293™ medium. The expression level of single mutants (K34D, K32A, K32D) and double mutants (K34D/K32A, K34D/K32D) were comparable to the wildtype.

FIG. 15 shows that NGF K34D dramatically reduces the binding affinity to p75^{NTR} while retaining binding to TrkA and NGF K34D/K32D eliminated the binding affinity to p75^{NTR} while retaining TrkA binding.

FIG. 16 shows the experimental design of the glaucoma model study in rats (n=8/group).

FIG. 17 shows that the intraocular pressure (IOP) is elevated in the left eyes prior to the treatments from 20 to 32 mmHg.

FIG. 18 shows that both NGF K34D and NGF K34D/K32D improve the pattern electroretinogram amplitude (indicator of retinal ganglion cell function) while the wildtype NGF has no effect.

FIG. 19 shows that the wildtype induces persistent mechanical allodynia throughout the 7 day period while the allodynia effect of NGF K34D/K32D is less persistent and becomes non-significant on days 4 and 7(n=10/group).

### DETAILED DESCRIPTION OF THE INVENTION

As used herein, the following definitions and abbreviations apply.

“Examplary” (or “e.g.” or “by example”) means a non-limiting example.

“Identity,” as used herein in reference to polypeptide or DNA sequences, refers to the subunit sequence identity between two molecules. When a subunit position in both of the molecules is occupied by the same monomeric subunit (i.e., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990). For example, sequence identity can be calculated using the default values for BLASTP and BLASTN indicated by Madden T. (Appendices. 2008 Jun. 23 [Updated 2021 Jun. 24]. In: BLAST® Command Line Applications User Manual [Internet]. Bethesda (MD): National Center for Biotechnology Information (US); 2008-. Available from: https://www.ncbi.nlm.nih.gov/books/NBK279684/, e.g. see blastp and blastn application options.). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof. For the avoidance of doubt, where sequence identity is defined between an instant NGF mutein and a referenced sequence, additional sequences in the instant NGF mutein outside of the referenced NGF wt sequence does not reduce the calculated identity.

“K34D” (or similarly, one letter amino acid designation followed by a number and a one letter amino acid designation) refers to wild type NGF mature protein where the first letter is the naturally occurring amino acid residue and the second letter is the substituted amino acid residue (or “mutated”).

“NGF” means mature nerve growth factor.

“Numbered in accordance with wild type NGF”, is meant to identify a chosen amino acid residue with reference to the position at which that residue normally occurs in the mature sequence of wild type human NGF (SEQ ID NO: 2).

“Ophthalmically acceptable” means having no persistent detrimental effect on the treated eye or the functioning thereof, or on the general health of the subject being treated. However, it will be recognized that transient effects such as minor irritation or a “stinging” sensation are common with topical ophthalmic administration of drugs and the existence of such transient effects is not inconsistent with the composition, formulation, or ingredient (e.g., excipient) in question being “ophthalmically acceptable” as herein defined. In some embodiments, the pharmaceutical compositions can be ophthalmically acceptable or suitable for ophthalmic administration.

“Pro-sequence” means a sequence N-terminal to a mature NGF (or NGF mutein) containing a proteolytic cleavage site; e.g., a site recognized by endogenous NGF-processing protease such as furin.

“p75^{NTR} bio-activity”(or “p75^{NTR} biological activity”) means an in vivo or in vitro biological activity that is mediated by p75^{NTR}. Optionally, p75^{NTR} bio-activity is p75^{NTR} mediated differentiation in a primary rat oligodendrocyte assay

“p75^{NTR} binding activity means physical binding to p75^{NTR}, for example, as measured in a BioCore 3000.

“TrkA bio-activity” (or “TrkA biological activity”) means an in vivo or in vitro biological activity that is mediated by TrkA. Optionally, the TrkA bio-activity is TrkA-mediated mitogenesis, for example, as demonstrated in TF1 erythroleukemic-based proliferation assay.

“TrkA binding-activity” means physical binding to TrkA, for example, as measured in a BioCore 3000.

“Wildtype NGF” means NGF, whether native or recombinant, having the 120 normally occurring amino acid sequence (SEQ ID NO: 2) of native human NGF (less the signal peptide and pro-protein peptide), unless otherwise stated.

“wt” means wildtype.

### NGF Muteins-Primary Structure

NGF muteins of the present invention have a high level of sequence identity with the mature form of human NGF (SEQ ID NO. 2) or an active fragment thereof (e.g. residues 1-117 or 1-118 of SEQ ID: NO. 2). By way of example, sequence identity is about 85% or more, or about 90% or more, or about 95% or more, or about 98 or 99% or more. Additionally, species of the instant NGF muteins have an acidic amino acid residue at one or both of amino acid residues 32 and 34, where residue numbers refer to mature human NGF of SEQ ID NO. 2. The acidic residue can be a natural amino acid residue such as aspartic acid or glutamic acid. The acidic residue can be an unnatural amino acid residue that is a derivative of aspartic acid such as N—Z—L-aspartic anhydride or 4-tert-butyl hydrogen 2-azidosuccinate. The acidic residue can be an unnatural amino acid residue that is a derivative of glutamic acid such as γ-carboxy-DL-glutamic acid.

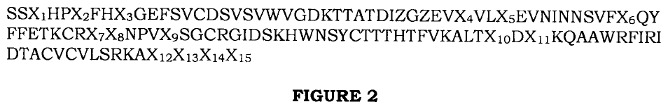

An instant NGF mutein can optionally comprise the sequence shown in FIG. 2 or an active fragment thereof (e.g. residues 1-117 or residues 1-118), wherein one or both of the Z amino acids (residues 32 and 34) are an acidic amino acid and wherein X1-X15 are any amino acid. Alternatively, the invention contemplates an NGF mutein having a sequence with 5 or fewer substitutions, insertions, or deletions relative to FIG. 2, wherein one or both of the Z amino acids are an acidic amino acid and wherein X1-X13 are any amino acid. Optionally, the NGF mutein has an N-terminal extension comprising one or more amino acid residues in a peptide bond to the core structure or a C-terminal extension comprising one or more amino acid residues in a peptide bond to the core structure, or a C-terminal and an N-terminal extension. Based on analysis of related homologs in humans, other species, and known active variants, the inventor believes residues X1-X15 are among the various residues that can be substituted while retaining one or more NGF functions taught herein. Optionally, each of X1-X15 is independently selected from the following a) any natural or unnatural amino acid residue; b) the amino acid present at the same position in human wildtype NGF or wildtype NGF of any other animal species; and c) an amino acid of the same class (non-polar neutral, polar neutral, acidic, basic) as the amino acid present at the same position in human wildtype NGF or wildtype NGF of any other species. Optionally, only one of the Z amino acids (residue 32 or 34) is acidic and the other Z amino acid is selected from a) the amino acid present at the same position in human wildtype NGF (Lys) or wildtype NGF of any other species; b) an amino acid of the same class (basic, e.g. Arg or His) as the amino acid present at the same position in human wildtype NGF or wildtype NGF of any other species; and c) a non-polar neutral amino acid such as alanine, glycine, isoleucine, leucine, methionine, phenylalanine, proline and valine. Additionally or alternatively, the invention contemplates embodiments in which any of X13-X15 are optionally absent (e.g. the NGF mutein has residues 1-117 or 1-118 of FIG. 2). The NGF mutein can have any of the properties or activities described herein (e.g. described directly below or properties A)-I) taught herein).

In another embodiment the instant NGF muteins have increased biological activity for TrkA (when compared to wild-type NGF) as demonstrated by a lower EC50 in a TF1 erythroleukemic cells proliferation assay (e.g. as demonstrated in Example 2). For example, In one embodiment, the EC50 for TrkA is of an instant NGF mutein is no greater than one of about 90%, or about 80% or about 50% or about 30% of wildtype NGF EC50 (see Table 1).

In one embodiment, the instant NGF muteins have reduced biologic activity towards p75^{NTR} (when compared to wild-type NGF) as demonstrated by at least a 20% reduction in differentiation using a primary rat oligodendrocytes assay (which express p75^{NTR}) as demonstrated, e.g. in Example 3. In one embodiment, the instant NGF mutein is designated here as “NGF K34D” (or “K34D”), which has 100% sequence identity with SEQ ID NO. 2 except that the lysine at residue number 34 is substituted with an aspartic acid.

Another one of the instant NGF muteins is designated here as “NGF K34E” (or “K34E”), which has 100% sequence identity with SEQ ID NO. 2 except that the lysine at residue number 34 is substituted with a glutamic acid.

In one embodiment, the instant NGF muteins are the proteins of paragraph [0053] further comprising a mutation at amino acid residue K32.

In one embodiment, the instant NGF muteins are the proteins of paragraph [0053] further comprising a mutation at amino acid residue K32D.

In embodiments comprising mutations in positions 32 and/or 34, as taught herein, the substitutions can optionally be any acidic amino acid and the sequence can optionally have 100% sequence identity with SEQ ID NO. 2 except for (i.e. not considering) the mutations at positions 32 and 34, or can optionally have greater than 80% sequence identity with SEQ ID NO. 2 except for (i.e. not considering) the mutations at positions 32 and 34, or can optionally have 5 or less substitutions relative to SEQ ID. NO 2 or FIG. 2.

### Examplary Amino Acid Substitutions

As taught herein, NGF muteins can comprise one or more acidic amino acid substitutions. The acidic amino acid can be any amino acid having a side chain that is acidic a neutral pH, e.g., a side chain having a carboxylic acid group, e.g. whose pKa is low enough to lose a proton at neutral pH. For example, the acidic amino acid can be any amino acid having a carboxylic acid side chain in which the deprotonated form of the carboxylic acid predominates at pH 7. The acidic residue can be a natural amino acid residue such as aspartic acid or glutamic acid. The acidic residue can be an unnatural amino acid residue that is a derivative of aspartic acid such as N—Z—L-aspartic anhydride or 4-tert-butyl hydrogen 2-azidosuccinate. The acidic residue can be an unnatural amino acid residue that is a derivative of glutamic acid such as γ-carboxy-DL-glutamic acid. Such an acidic amino acid can be provided at position 32 and/or 34.

Alternatively, the invention contemplates any amino acid substitution in which the amino acid comprises a side chain that carries (or predominantly carries) a negative charge at pH 7. Such an amino acid can be provided at position 32 and/or 34.

The amino acid substitutions described above (e.g. at position 32 and/or 34) can be natural or unnatural. Examples of natural amino acids include aspartic acid or glutamic acid. Examples of unnatural amino acids include D-amino acids, Homo-amino acids, Beta-homo-amino acids, N-methyl amino acids, Alpha-methyl amino acids, and gamma amino acids. Such an unnatural amino acid can optionally be an analog of a natural amino acid, e.g. the same as aspartic acid or glutamic acid with the exception of the modification (e.g. insertion of a methylene group to the a-carbon in the case of Homo-amino acids).

### NGF Mutein Fragments

For the sake of illustration, the NGF muteins are generally described herein with respect to residues 1-120 of SEQ ID NO. 2 or FIG. 2. However, fragments of these sequences are also useful according in the present invention so long as they exhibit NGF activity such as the ability to stimulate TrkA bio-activity. For example, fragments of wildtype NGF are known to provide peptides with the same or similar activity to the 120-residue wildtype NGF, for example, NGF1-117 and NGF1 118 (see for example WO1997017087A1 to Young et al., and NGF1-118 sold under name OXERVATE®) are active fragments. Accordingly, the invention also contemplates active fragments of the NGF muteins taught herein, for example, a fragment having 1, 2, or 3 of the C-terminal amino acids of SEQ ID NO 2 or FIG. 2 removed (e.g. an NGF mutein fragment comprising residues 1-117 or 1-118 of SEQ ID NO 2 or FIG. 2). The fragment can be, for example, any fragment having one or more properties of various NGF muteins described herein, such as, relative to wildtype NGF, reduced or eliminated p75NTR binding, enhanced TrkA bioactivity, retained or increased in thermostability, reduced pain upon administration to a patient (e.g. to the eye), ability to dimerize with itself or another NGF, and/or exhibition of disulfide bonds and forms a cysteine knot structure. Similarly, the invention also contemplates NGF muteins having high homology (e.g. greater than 80% or greater than 90% sequence identity) to such fragments of SEQ ID NO: 2 and FIG. 2.

### Fusions and Conjugates

Instant NGF muteins can be provided as a fusion or conjugate with a moiety such as a peptide or small molecule, polymer, etc. NGF fusions and conjugates can be provided by expressing a gene encoding the fusion or by chemical conjugation to the moiety.

Examples of moieties that can be fused or conjugated with NGF include blood brain barrier transfer agents such as transferrin (e.g. as disclosed by CN1876682A), polyethylene glycol (e.g. as disclosed by CN102964442A), drugs (e.g. as disclosed by WO1995001806A1), transporting agents (e.g. as disclosed by WO2005009394A2), and moieties conferring increased half-life such as immunoglobin Fc or albumin (e.g. as disclosed by WO2001077137A1 and U.S. Pat. No. 6,946,134). Another example is a moiety which can bind a phase used purification or otherwise enhances purification, such as Fc or albumin (e.g. as disclosed U.S. Pat. No. 6,946,134), for example, IgG Fc which allows binding to Protein A, and albumin which binds to Blue Sepharose Column.

In one embodiment, the invention is a conjugate or fusion protein comprising an instant NGF mutein (set forth above) and another moiety, wherein the moiety is a therapeutic agent, a targeting agent, or a modifier to confer a physicochemical property to the NGF mutein protein (e.g. increased half-life). The fusion or conjugation can occur at the amino-terminal or carboxy-terminal amino acid residue of a peptide moiety and can be coupled (e.g. through a peptide bond) to either terminal of the NGF mutein.

Optionally, the moiety can be a mutated peptide. For example, where the structural moiety is the Fc domain, this domain can comprise one or more amino acid substitutions that reduce binding to Fc receptor and antibody-dependent cell-mediated cytotoxicity.

### Production of the Instant Invention

Instant NGF muteins can be produced by a protein synthesizer or by using recombinant techniques. Recombinant techniques for such proteins are well known in the art at this time and the recombinant production may be conducted in variety of cells and settings, including animal, plant and microbial cells in culture and as an in situ location in multicellular organisms.

Instant NGF muteins can be produced by creating an expression vector with the appropriate polynucleotide sequence to produce the instant muteins and transfecting or transforming a host cell population with the expression vector.

When the host cell is a eukaryotic cell, it is useful to precede the mutein coding sequence with a signal sequence that allows the mutein to be inserted in the lumen of the endoplasmic reticulum. While the signal sequence can be any signal sequence, the inventors have discovered that mouse Ig kappa signal sequence is especially useful.

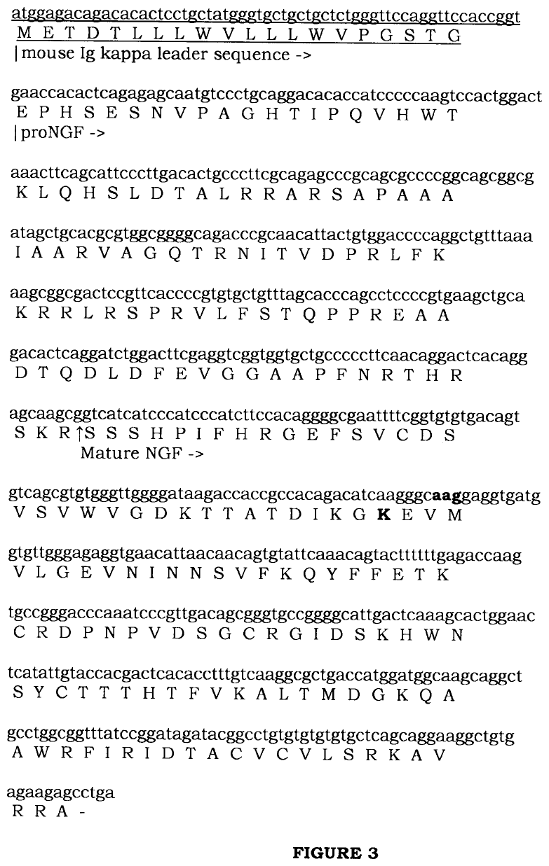

It has also been discovered that inserting a pro-sequence after the leader sequence and before the instant mutein sequence can result in increased production of biologically active muteins. An instant pro-sequence is a sequence that is cleavable by endogenous protease of the instant host cell or by a protease added to the host cell culture medium. An examplary signal sequence/pro-sequence/mutein sequence construct is shown in FIG. 3.

Instant host cells can be any host cells that can be transfected or transformed with a sequence coding for an instant NGF mutein. The inventors have discovered that eukaryotic cells are especially useful to produce biologically active muteins of the instant invention. Mammalian cells and particularly human cells are useful. When constructs of the present invention include a signal sequence and a pro-sequence, host cells that co-express pro-sequence processing protease(s) (either naturally or by transformation) are especially useful. Furin is one such pro-sequence processing protease that is especially useful according to the present invention.

It has been discovered that mammalian host cells transformed or transfected with constructs containing a signal sequence and a pro-sequence can produce high-yield, biologically active NGF muteins of the present invention with a highly homogeneous N terminus, making it especially useful for meeting the requirements of governmental drug regulating administrations.

HEK293 cells are particularly suitable for the production due to efficient processing of pro-NGF into mature NGF (i.e. NGF beta). The invention therefore includes recombinant materials for production of these components, such as expression systems with suitable control sequences, vectors, host cells harboring these and the like.

Other useful methods of producing the NGF mutein are disclosed by EP0786520A1.

### Indications

The instant NGF muteins and pharmaceutical compositions thereof are useful, for example, for treating any disease, for example, diseases of the central nervous system such as stroke, depression, traumatic brain injury, spinal cord injury, Parkinson's disease, Alzheimer's disease, Huntington's disease, optic glioma and advanced optic nerve atrophy, hypoxic-ischemic perinatal brain injury, or Amyotrophic Lateral Sclerosis.

The instant muteins and pharmaceutical compositions thereof are useful in the treatment of skin trauma and/or diseases such as lower limb crush syndrome, pressure ulcers, vasculitic ulcers, diabetic foot ulcers, corneal ulcers, and other skin ulcers.

Instant proteins (NGF muteins) and compositions thereof are useful to treat subjects with ophthalmologic pathologies such as neurotrophic keratitis, glaucoma, bilateral age-related macular degeneration (retinopathy). Without being bound by theory, the inventors believe that administration of the instant invention to such subjects protect retinal cells from degeneration and apoptosis in glaucoma and can improve visual function in patients with glaucoma.

Instant proteins and compositions thereof are useful for treatment for dry eye. NGF is presently being evaluated for treatment of dry eye (Sacchetti M, et al Br J Ophthalmol. 104:127, 2020). However, the present NGF muteins, with enhanced therapeutic activity and/or reduction of pain provide surprisingly superior efficacy.

Other indications for the instant muteins and pharmaceutical compositions are peripheral sensory neuropathies, e.g. diabetic polyneuropathy, HIV-associated peripheral neuropath, and chemotherapy-induced peripheral neurotoxicity.

The NGF muteins and pharmaceutical compositions taught herein can be used to promote the migration of fibroblasts in a subject in need thereof, for example, to promote wound healing. Examples of wounds treatable by the instant muteins include skin wounds, wounds induced by surgery (e.g. scalpel wounds), diabetic wounds and ulcers (e.g. foot or leg ulcers). Such an NGF mutein can be administered systemically or topically.

The NGF muteins taught herein can be administered to treat a condition of the eye. For example, instant NGF muteins can be administered to the ocular surface (e.g. topically), to treat conditions of other eye such as intraocular tissue pathologies of, for example, the sclera, ciliary body, crystalline lens, retina, optic nerve, vitreous body and choroidea, e.g. as disclosed by U.S. Pat. No. 10,010,586. Optionally, the NGF muteins can be administered topically, and penetrate through ocular tissue to provide therapy to the retina, optic nerve, or against any affections involving the above reported internal structures of the eye. Optionally, the NGF muteins are administered to the eye at a concentration greater than (20 μg/μl ), e.g. with reduced pain relative to wildtype NGF.

The NGF muteins and pharmaceutical compositions taught herein can be administered to treat any wound. Example of wounds include corneal wounds, surgical wounds (e.g. from cataract and refractive surgery).

The NGF muteins and pharmaceutical compositions taught herein can be administered to prevent neuron damages of surgical, chemical, mechanical and ischemic origin. For example, such activity can be used for the therapy of various pathologies affecting either or both of the peripheral and central nervous systems (see, for example, Hefti F., J. Neurobiol., 25:1418, 1994; J. Fricker, Lancet, 349:480, 1997).

The NGF muteins and pharmaceutical compositions taught herein can be administered to prevent hearing loss. For example, US 2020/0155647 describes the use of neurotrophins in the treatment of hearing disorders related to hearing loss. The skilled artisan will recognize these and other disorders which can be treated by the instant NGF muteins.

The NGF muteins taught herein can be used to modulate PI3K/Akt, Rac1, JNK, and/or ERK activity in a patient in need thereof. Many indications associated with such activity are known in the art. Such activity can optionally be activity involved in the NGF-induced fibroblast migration (e.g. as in wound healing).

NGF muteins of the present invention (e.g. comprising acidic amino acid substitutions at residues 32 and 34) can be administered to patients to provide high efficacy yet reduced pain relative to wildtype NGF. NGF administration is known to induce substantial pain, intolerable in some cases such as Alzheimer's disease and diabetic neuropathy, in both of which NGF is administered parenterally. For example, a phase I clinical trial of NGF for Alzheimer's disease was discontinued due to intolerable pain. In phase I-III clinical trials of NGF for diabetic neuropathy, significant pain was still an issue, with a significant number of patients dropping out due to pain, and requiring limited dosing in an effort to curb this side effect. Accordingly, it is quite surprising that NGF muteins of the present invention can be administered to treat diseases with high efficacy and reduced pain. Though insight of the inventor, NGF muteins have been discovered (e.g. comprising acidic amino acid substitutions at residues 32 and 34) which can be administered to treat various diseases with substantially reduced pain relative to wildtype NGF.

The invention also provides the use of an NGF mutein or composition taught herein to treat any of the indications taught herein.

Use of NGF muteins of the present invention are not particularly limited to any of the foregoing indications. The NGF muteins can be used to stimulate differentiation, survival, and growth of neurons in any patient in need thereof. For example, the NGF muteins can be used treat or prevent any disease or condition associated with death of neurons or decline of neuronal signaling such as dementia, senile dementia, memory loss, or aging. For example, the NGF muteins can be used to maintain healthy aging, e.g. administered to prevent the onset of age-related conditions such as dementia, Alzheimer's, loss of memory, or loss of cognitive function.

Surprisingly, it has been discovered that an NGF mutein of the invention exhibits increased concentration and/or half-life of the NGF mutein in clinically important anatomical regions (e.g. optical nerve or sclera) relative to wild type NGF. For example, this can be demonstrated by administering an NGF mutein or wt NGF in the same manner (e.g. topically such as to the eye) (e.g. in a composition of 200 μg/ml of NGF) and measuring the amount or concentration of NGF (wild type or mutein, or active fragment thereof, or active metabolic product thereof) in a specified anatomical region. Surprisingly, the NGF mutein exhibits increased concentration, relative to wt NGF, in clinically important anatomical regions at 2 hours, 6 hours, and/or 24 hours following the administration. Surprisingly, such an NGF mutein now allows for once-a-day treatment (e.g. topical administration to the eye). Other examples of clinically important anatomical regions in which an NGF mutein of the invention can accumulate following administration (e.g. topically to the eye) include ciliary bodies, sclera, retina, lens, vitreous bodies, optic nerves, choroid, corneas, irises, or anterior humor.

### Administering Compositions of the Invention In Vivo

The compositions of the present invention may be administered to humans and other mammals as well as to domestic avian species. These may include companion or farm animals, for example. For treatment of human ailments, a qualified physician can determine how the compositions of the present invention should be utilized with respect to dose, schedule and route of administration using established protocols. A similar role is assumed by a veterinarian in other species. Such applications may also utilize dose escalation.

The pharmaceutical compositions of the present invention can be administered topically or parenterally, e.g., via eye drops, nasal spread, intraarterially, intravenously, intraperitoneally, subcutaneously, or intramuscularly. For example, topical pharmaceutical compositions can be administered as eye drops or nasal spray.

As taught herein, embodiments of an NGF mutein taught herein (e.g. comprising mutations at residue 32 and 34) can provide superior efficacy with reduced pain relative for wildtype. Pain induction has been a major problem of parenteral administration of wildtype NGF-induced pain, creating issues for both dosing and patient compliance, even more particularly for long-term treatment. Even for topical administration of low concentrations of NGF (Oxervate; 20 μg/μl ), pain is still the most frequently reported adverse event (16% of patients). Accordingly, through insight of the inventor, particular embodiments of NGF muteins can be administered parenterally (e.g. intravenously or subcutaneously) or topically while providing superior efficacy, reduced pain, and greater patient compliance, relative to wildtype. Moreover, the NGF muteins can be administered over the long term (e.g. longer than 2 weeks) while providing such advantages.

The NGF muteins and pharmaceutical compositions can be administered to treat human cutaneous pressure ulcer, corneal ulcers, glaucoma, retinal maculopathy, Retinitis Pigmentosa, pediatric optic gliomas and brain traumas. The NGF muteins and pharmaceutical compositions can be used to treat any condition known or suggested to be treatable with NGF, e.g. as disclosed by Rocco (Nerve Growth Factor: Early Studies and Recent Clinical Trials. Curr Neuropharmacol. 2018;16(10):1455-1465).

The pharmaceutical compositions of the present invention can also be administered transmucoally, e.g. Intranasal, oral transmucosal, intra vaginal mucosal, oral, rectal, and intracerebroventricular routes.

### Pharmaceutical Compositions

The instant NGF muteins can be formulated in any pharmaceutically acceptable composition.

Pharmaceutical compositions of the invention are prepared according to standard techniques and may comprise water, buffered water, saline, glycine, dextrose, iso-osmotic sucrose solutions and the like, including glycoproteins for enhanced stability, such as albumin, lipoprotein, globulin, and the like. These compositions may be sterilized by conventional, well-known sterilization techniques. The resulting aqueous solutions may be packaged for use or filtered under aseptic conditions and lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and the like.

The concentration of the invention components in the pharmaceutical formulations can vary widely, such as from less than about 1 μg/ml, e.g., at or at least about 100 μg/ml to as much as 10 mg/ml by weight and will be selected primarily by fluid volumes, viscosities, and the like, in accordance with the particular mode of administration selected. For example, the concentration may be increased to lower the fluid load associated with treatment.

The pharmaceutical compositions can be configured for topical application, e.g. as creams, ointments, gels, lotions, sprays, powders, aerosols, liniments, and drops.

The pharmaceutical compositions of the present invention can be administered topically as an eye drop, nasal spread, or hydrogel. The treatment also can be administered via intraocular, intravenous, subcutaneous, or intramuscular routes. Dosage for the delivery vehicle formulations will depend on the ratio of drug to lipid and the administrating physician's opinion based on age, weight, and condition of the patient.

In addition to pharmaceutical compositions, suitable formulations for veterinary use may be prepared and administered in a manner suitable to the subject. Preferred veterinary subjects include mammalian species, for example, non-human primates, dogs, cats, cattle, horses, sheep, and domesticated fowl. Subjects may also include laboratory animals, for example, in particular, rats, rabbits, mice, and guinea pigs.

A pharmaceutical composition can be formulated as an ophthalmic composition, for example, an eye drop. The ophthalmic composition is optionally an aqueous ophthalmic composition, a non-aqueous ophthalmic composition, a lyophobic ophthalmic composition and a lyophilic ophthalmic composition.

Optionally, the ophthalmic composition configured for administered via a contact lens, e.g. configured such that the composition is has similar physical properties as an intraocular liquid or an aqueous humor. The ophthalmic composition can comprise an isotonicity-imparting agent and/or a buffer.

Optionally, the ophthalmic composition comprises one or more of glucose, sodium chloride, potassium chloride, calcium chloride, magnesium sulfate, sodium hydrogen carbonate, and glutathione.

Optionally, the ophthalmic composition comprises an aqueous solvent such as sterilized purified water, physiological saline, and/or a solvent containing various kinds of components configured for an ophthalmic solution, e.g. electrolytic ions such as BSS plus (trade name, produced by Alcon Co.), a buffer, an isotonicity-imparting agent, glutathione, glucose and/or a solvent containing vitamin B12.

Optionally, the ophthalmic composition comprises a non-aqueous solvent, for example, a vegetable oil such as cottonseed oil, soybean oil, sesame oil, peanut oil, castor oil, olive oil, camellia oil, rapeseed oil, corn oil, or fluid paraffin.

Optionally, the ophthalmic composition comprises an isotonicity-imparting agent such as sodium chloride, boric acid, potassium nitrate, D-mannitol, and/or glucose. For example, the isotonicity-imparting agent be present in an amount that provides an osmotic pressure ratio of 0.6 to 2.0.

Optionally, the ophthalmic composition comprises a pH adjuster, for example, boric acid, anhydrous sodium sulfite, hydrochloric acid, sodium hydroxide, sodium citrate, acetic acid, potassium acetate, sodium carbonate, sodium hydrogen carbonate, borax, and/or a buffer (e.g., a citrate buffer, a phosphate buffer, etc.). Optionally, the pH adjuster is present in an amount that provides a pH of about 3.0 to 8.0.

Optionally, the ophthalmic composition comprises a viscosity-imparting agent, e.g., methyl cellulose, hydroxypropyl cellulose, hydroxypropylmethyl cellulose, polyvinyl alcohol, sodium chondroitin sulfate, and/or polyvinyl pyrrolidone. Optionally, the viscosity-imparting agent is present in an amount that provides a viscosity enough to be dropped from an ophthalmic solution bottle can be imparted, and it may be, for example, 0.001% to 10% (W/V).

Optionally, the ophthalmic composition comprises a suspending agent, e.g., Polysorbate 80 (trade name), polyoxyethylene hardened castor oil, polyoxy hardened castor oil, and/or carboxymethyl cellulose. Optionally, the suspending agent is present in an amount of about 0.001% to 10% (W/V).

Optionally, the ophthalmic composition comprises an emulsifier, e.g., yolk lecithin and/or Polysorbate 80. Optionally, the emulsifier is present in at 0.001% to 10% (W/V).

Optionally, the ophthalmic composition comprises a preservative, e.g., benzalkonium chloride, benzethonium chloride, chlorobutanol, phenylethyl alcohol, and/or paraoxybenzoate. Optionally, the preservative is present in at 0.001% to 10% (W/V).

Optionally, the ophthalmic composition comprises the NGF mutein at a concentration of about 0.5 μg/ml to about 1000 μg/ml. For example, useful concentrations include any of about 0.5 μg/ml to about 500 μg/ml; about 0.5 μg/ml to about 200 μg/ml; about 1 μg/ml to about 500 μg/ml; about 1 μg/ml to about 200 μg/ml; about 1 μg/ml to about 100 μg/ml; about 0.5 μg/ml to about 50 μg/ml; about 1 μg/ml to about 50 μg/ml; about 20 μg/ml to about 1000 μg/ml; about 50 μg/ml to about 1000 μg/ml; about 100 μg/ml to about 1000 μg/ml, at least about 100 μg/ml, at least about 200 μg/ml, or at least about300 μg/ml.

Optionally, the ophthalmic composition comprises the NGF mutein at a concentration of about 0.0001 to 0.5% (W/V).

Optionally, the composition comprises the NGF mutein as a dimer such as a homo-dimer, e.g. wherein a majority or substantially all of the NGF mutein is present as a dimer such as a homo-dimer.

As taught herein, reduction of pain, relative to wildtype NGF, is a benefit of various NGF muteins of the present invention. One of the most common adverse reactions in patients taking ophthalmic NGF at 20 μg/ml, sold under the name Oxervate™, is pain, which is not only difficult for the patient to endure but also reduces patient compliance with administration protocols. Accordingly, it is surprising that the NGF muteins of the present invention (e.g. having acidic amino acids at residues 32 and 34) can reduce pain and increase patient compliance while treating conditions that can be mediated by NGF. Thus, the compositions of the present invention can be provided at concentrations of 20 μg/ml or greater to provide greater NGF therapeutic activity (e.g. sufficient to treat glaucoma with enhanced efficacy) with surprisingly low pain-related adverse reactions and surprisingly high patient compliance, but can also be provided at contraptions of no more than 20 μg/ml to provide extremely low or even completely eliminated pain and extremely high patient compliance, while treating, for example, indications for which wildtype NGF or Oxervate is currently used clinically.

The pharmaceutical composition can optionally comprise other useful ingredients in combination with the NGF mutein, such as those disclosed by U.S. Pat. No. 7,074,763.

### Properties of the Instant NGF Muteins

Genera of NGF muteins are taught throughout this application. For example, the NGF mutein can have a high sequence homology to SEQ ID NO. 2 and include substiutions (e.g. of amino acids having acidic or negative side chains) at residues 32 and/or 34. FIG. 2 represents a subgenus of such an embodiment. With the teachings provided herein, the skilled artisan will appreciate that various amino acid selections or substitutions, conjugates, and N-terminal or C-terminal fusions can be made while providing for one or more properties or advantages taught herein. Such properties and advantages include, for example, the following.

A) An instant NGF mutein can provided which has increased biological activity for TrkA (when compared to wild-type NGF) as demonstrated by a lower EC50 in a TF1 erythroleukemic cells proliferation assay (e.g. as demonstrated in Example 2), e.g. the EC50 is of an instant NGF mutein is no greater than one of about 90%, or about 80% or about 50% or about 30% of wildtype NGF EC50about 20% lower or about 50% lower or about 100% lower.

B) An instant NGF mutein can provided which has reduced biologic activity towards p75^{NTR} (when compared to wild-type NGF) as demonstrated by at least a 20% reduction in differentiation using a primary rat oligodendrocytes assay (which express p75^{NTR}) as demonstrated, e.g. in Example 3. Optionally, EC50 is of an instant NGF mutein for such activity is no greater than one of about 90%, or about 80% or about 50% or about 30% of wildtype NGF EC50 about 20% lower or about 50% lower or about 100% lower. Such an NGF mutein can optionally also exhibit feature A).

C) An instant NGF mutein can provided which induces less pain than wildtype NGF or Oxervate, e.g. when administered topically to the eye of a patient (e.g. at 200 μg/ml ) or parenterally (e.g. IV or subcutaneously). Such an NGF mutein can optionally also exhibit feature A).

D) An instant NGF mutein can provided which dimerizes with itself.

E) An instant NGF mutein can provided which exhibits three disulfide bridges and forms a cysteine knot.

F) An instant NGF mutein can exhibit A), B), and C) and optionally J).

G) An instant NGF mutein can exhibit A), B), C), and E) or A), B), C), D), and E) and optionally J) or K).

H) An instant NGF mutein can provided which exhibits greater efficacy in treating any condition taught herein, optionally in combination with any of features A-G, I, J, and K

I) An instant NGF mutein can provided which exhibits any of features A)-H), and retained or increased thermal stability relative to wildtype NGF.

J) An instant NGF mutein can be provided which exhibits an increase in biological activity towards TrkA, relative to wildtype NGF (i.e. a positive change) and a decrease in biological activity towards p75^{NTR} (i.e. a negative change). Alternatively, an instant NGF mutein can be provided which exhibits a decrease in biological activity towards TrkA, relative to wildtype NGF (i.e. a negative change) and a decrease in biological activity towards p75^{NTR} (i.e. a negative change) wherein the change in biological activity towards TrkA (e.g. determined as in Example 2) is less than the change in biological activity towards p75^{NTR} (e.g. determined as in Example 3). Accordingly, even for NGF muteins which exhibit a decrease in TrkA, the greater decrease in activity towards p75^{NTR} outweighs, in terms of net therapeutic efficacy, any loss in TrkA activity. For example, in the instance in which both TrkA and p75^{NTR} activity increase, the change in the respective activities can optionally be determined by the EC50 of wildtype divided by the EC50 of the NGF mutein. In such an illustrative example the increase in TrkA activity of the NGF mutein, relative to wildtype NGF (e.g., calculated as [EC50 wildtype NGF for TrkA activity]/[EC50 NGF mutein for TrkA activity]) is greater than the increase in p75^{NTR} activity of the NGF mutein, relative to wildtype NGF (e.g. calculated as [EC50 wildtype NGF for p75^{NTR} activity]/[EC50 NGF mutein for p75^{NTR} activity]); in other words ([EC50 wildtype NGF for TrkA activity]/[EC50 NGF mutein for TrkA activity]) is greater than ([EC50 wildtype NGF for p75^{NTR} activity]/[EC50 NGF mutein for p75^{NTR} activity]).

K) An instant NGF mutein can be provided which exhibits an improved therapeutic index compared to the wildtype NGF, as measured its ratio of EC₅₀ for pain vs EC₅₀ for RGC-protective efficacy.

Additionally, the present NGF muteins can exhibit any of the above properties relative to any other NGF protein referenced herein (e.g. Oxervate or prior art mutants).

As taught herein, embodiments of the present invention induce less pain than other NGF proteins such as wildtype NGF, Oxervate, and other NGF proteins referenced herein. Pain can optionally be measured as an ipsilateral 50% response threshold, as taught herein (see Examples). Optionally the pain is eye pain. Optionally, pain, such as eye pain (e.g. ocular pain) is scored or measured, e.g., by the Wong-Baker FACES® Pain Rating Scale or the Ocular Pain Assessment Survey (OPAS) (Qazi, Yureeda et al. “Validity and Reliability of a Novel Ocular Pain Assessment Survey (OPAS) in Quantifying and Monitoring Corneal and Ocular Surface Pain.” Ophthalmology vol. 123,7 (2016): 1458-68. doi:10.1016/j.ophtha.2016.03.00). Other methods of measuring pain are well known in the art. Optionally, pain, such as eye pain is quantified as the number of pain or pain-related adverse events in a population (e.g. 50 or more or 100 or more) of subjects (e.g. glaucoma patients) to which the NGF mutein is administered (e.g. at 200 μg/ml ). For example, the most common adverse reaction reported in clinical trials of ophthalmic Oxervate® (20 μg/μl ) is eye pain, which occurred in 16% of patients (Oxervate® (cenegermin-bkbj) ophthalmic solution 0.002% (20 mcg/mL)[US package insert]. Boston, Mass.; Dompé U.S. Inc.; 2019.; Bonini S, Lambiase A, Rama P, Sinigaglia F, Allegretti M, Chao W, Mantelli F, for the REPARO Study Group. Phase II randomized, double-masked, vehicle-controlled trial of recombinant human nerve growth factor for neurotrophic keratitis. Ophthalmology. 2018 Sep;125(9):1332-1343.) Certain embodiments of the present invention (e.g. an NGF mutein having acidic amino acids at residues 32 and 34) illicit fewer pain adverse events when administered at the same concentration.

### EXAMPLARY EMBODIMENTS

Among the various embodiments taught herein are the following exemplary embodiments (EEs).

1. An isolated human nerve growth factor (NGF) mutein comprising an acidic amino acid residue substitution at position 34 relative to SEQ ID NO. 2 and at least 80% sequence identity to SEQ ID NO. 2 or a fragment thereof that activates TrkA signaling or stimulates TrkA bioactivity.
2. The NGF mutein of EE 1 wherein the acidic amino acid residue substitution is aspartic acid, glutamic acid, N—Z—L-aspartic anhydride, 4-tert-butyl hydrogen 2-azidosuccinate, or γ-carboxy-DL-glutamic acid.
3. The NGF mutein of EE 1 wherein the acidic amino acid residue substitution is an aspartic acid.
4. The NGF mutein of EE 1 wherein the acidic amino acid residue substitution is a glutamic acid.
5. The NGF mutein of EE 1 further comprising a substitution at position 32 with an acidic amino acid.
6. The NGF mutein of EE 5, wherein the substitution at position 32 is an aspartic acid, glutamic acid, N—Z—L-aspartic anhydride, 4-tert-butyl hydrogen 2-azidosuccinate, or γ-carboxy-DL-glutamic acid.
7. The NGF mutein of EE 1 having the structure of FIG. 2 or an amino acid sequence differing from the structure of FIG. 2 by 5 substitutions or less among the amino acids other than X1-X15 and Z, wherein each of X1- X12 is any natural or unnatural amino acid residue and wherein one or more of the Z are any acidic amino acid residue, and wherein each of X13-X15 is any natural or unnatural amino acid residue or is absent.
8. The NGF mutein of EE 1 wherein said mutein has at least 30% of the biological activity towards TrkA when compared to a molar equivalent amount of wild type NGF.
9. The NGF mutein of EE 8 wherein the increased biological activity towards TrkA can be demonstrated by an EC50 in a TF1 erythroleukemic cell-based proliferation assay of not more than 170% of the EC50 of wild type NGF.
10. The NGF mutein of any of EEs 1-9 wherein the mutein has reduced p75NTR biologic activity, optionally as demonstrated by at least a 20% increase in EC50 in a primary rat oligodendrocyte differentiation assay.
11. The NGF mutein of any of EEs 1-9 wherein the mutein has reduced p75NTR binding activity, optionally as demonstrated by a 100% increase in Kd for binding to p75^{NTR} in a Biocore 3000 determination.
12. The NGF mutein of any of EEs 1-9 wherein the mutein has reduced p75NTR binding activity and reduced p75NTR biologic activity.
13. The NGF mutein of any one of the preceding EEs wherein the mutein has at least 90% identity with the sequence of FIG. 2.

14. The NGF mutein of EE 2 , wherein the mutein, relative to wildtype NGF,
a. has reduced biological activity towards p75^{NTR} that can demonstrated by at least a 50% reduction in ° differentiation in a primary rat oligodendrocyte assay;
b. has reduced binding activity towards p75^{NTR} as demonstrated by a 100% increase in Kd for binding to p75^{NTR} as determined by BioCore3000;
c. retains at least 30% TrkA biological activity as demonstrated by a human TF1 erythroleukemic cell-based proliferation assay;
d. has at least a 2-fold increase in the ratio of TrkA binding affinity over p75^{NTR} binding affinity; and
e. optionally, the mutein further comprises a substitution at position 32.
   15. The NGF mutein of any one of the preceding EEs wherein when expressed in a eukaryotic cell expression system, expression levels are at least 30% of the expression levels of NGF WT in an otherwise identical expression system.
   16. An isolated polynucleotide comprising a DNA sequence that encodes the NGF mutein of any of the preceding EEs.
   17. An expression vector comprising the polynucleotide of EE 16.
   18. The expression vector of EE 17 further comprising a polynucleotide coding for a pro-sequence.
   19. The expression vector of EE 17 further comprising a polynucleotide coding for a leader sequence.
   20. The expression vector of EE 18 further comprising a polynucleotide coding for a leader sequence.
   21. An isolated host cell transformed with the expression vector of any one of EEs 17-20.
   22. The host cell of EE 21 wherein the host cell is a eukaryotic host cell.
   23. The host cell of EE 22 wherein the eukaryotic host cell is a cell is a mammalian cell.
   24. The host cell of EE 23 wherein the mammalian host cell is a human host cell.
   25. The host cell of EE 24 wherein the human host cell is from a HEK293 cell line.
   26. The host cell of EE 24 wherein the human host cell is from a HEK293 cell line cultivated in Expi293 medium .
   27. A pharmaceutical composition comprising an NGF mutein of any one of EEs 1-15 or an NGF produced by the host cell of any of EEs 21-26, in combination with a pharmaceutically acceptable carrier.
   28. The pharmaceutical composition of EE 27 wherein the composition is ophthalmically acceptable.
   29. A method of treating a subject in need thereof comprising administering to the subject an effective amount of the composition of EE 27 or 28.
   30. The method of EE 29 wherein the subject in need experiences central nervous system pathology.
   31. The method of EE 30 wherein the central nervous system pathology is stroke, depression, traumatic brain injury, spinal cord injury, Parkinson's disease, Alzheimer's disease, Huntington's disease, optic glioma, advanced optic nerve atrophy, hypoxic-ischemic perinatal brain injury, or amyotrophic Lateral Sclerosis, or a combination thereof.
   32. The method of EE 29 wherein the subject in need experiences skin trauma prior to said administration.
   33. The method of EE 32 wherein the skin trauma is lower limb crush syndrome, pressure ulcers, vasculitic ulcers, diabetic foot ulcers, corneal ulcers, or a combination thereof.
   34. The method of EE 29 wherein the subject in need experiences an ophthalmologic pathology prior to said administration.
   35. The method of EE 34 wherein the ophthalmologic pathology is neurotrophic keratitis, keratoconjunctivitis sicca, glaucoma, bilateral age-related macular degeneration, retinopathy, or a combination thereof.
   36. The method of 29 wherein the subject in need experiences neurological pathology prior to said administration.
   37. The method of any one of EEs 29-36 wherein said administering is parenteral, topical, IV, or subcutaneous administering.
   38. A method to produce the NGF mutein of any one of EEs 1-15 comprising culturing the host cells of any one of EEs 21-25 and recovering the NGF mutein from the culture.
   39. The composition of EE 28, wherein the concentration of the NGF mutein is greater than 20 μg/ml.
   40. The composition of EE 39, wherein the concentration of the NGF mutein is greater than 50 μg/ml.
   41. The composition of EE 39, wherein the concentration of the NGF mutein is greater than 100 μg/ml.
   42. The composition of EE 39, wherein the concentration of the NGF mutein is greater than 150 μg/ml.
   43. The composition of EE 28, wherein the concentration of the NGF mutein is less than 20 μg/ml or less than than 5 μg/ml.
   44. A method of treating a condition of the eye comprising administering the composition of any of EEs 28 and 39-43 topically to the eye of a subject in need thereof.
   45. The method of EE 44, wherein the administration comprises daily, weekly, or a plurality of times per week administration for greater than two weeks.
   46. The method of EE 44, wherein the administration comprises daily, weekly, or a plurality of times per week administration for greater than two months.
   47. A method of treating a condition comprising administering the NGF mutein of any one of EEs 1-15 parenterally to a subject, optionally wherein the administration is intravenous (IV) or subcutaneous.
   48. The method of EE 47, wherein the condition is selected from Alzheimer's disease and diabetic neuropathy.
   49. The method of EE 47, wherein the administration comprises daily, weekly, or a plurality of times per week administration for greater than two weeks.
   50. The method of EE 47, wherein the administration comprises daily, weekly, or a plurality of times per week administration for greater than two months.
   51. A method of treating a condition comprising administering the NGF mutein of any one of EEs 1-15, wherein the condition is selected from stroke, depression, traumatic brain injury, spinal cord injury, Parkinson's disease, Alzheimer's disease, Huntington's disease, optic glioma and advanced optic nerve atrophy, hypoxic-ischemic perinatal brain injury, or Amyotrophic Lateral Sclerosis.
   52. A method of treating a condition comprising administering the NGF mutein of any one of EEs 1-15, wherein the condition is selected from skin trauma and/or diseases such as lower limb crush syndrome, pressure ulcers, vasculitic ulcers, diabetic foot ulcers, corneal ulcers, and other skin ulcers.
   53. A method of treating a condition comprising administering the NGF mutein of any one of EEs 1-15, wherein the condition is selected from ophthalmologic pathologies such as neurotrophic keratitis, glaucoma, bilateral age-related macular degeneration (retinopathy).
   54. A method of treating a condition comprising administering the NGF mutein of any one of EEs 1-15, wherein the condition is dry eye.
   55. A method of treating a condition comprising administering the NGF mutein of any one of EEs 1-15, wherein the condition is selected from peripheral sensory neuropathies, e.g. diabetic polyneuropathy, HIV-associated peripheral neuropath, and chemotherapy-induced peripheral neurotoxicity.
   56. A method of treating a promoting fibroblast migration comprising administering the NGF mutein of any one of EEs 1-15 to a subject in need thereof.
   57. A method comprising administering the NGF mutein of any one of EEs 1-15 to treat or prevent neuron damage of surgical, chemical, mechanical or ischemic origin.
   58. A method of stimulating differentiation, survival, and/or growth of neurons comprising administering the NGF mutein of any one of EEs 1-15 to a patient in need thereof.
   59. An NGF mutein comprising an active fragment of the NGF mutein of EE 7.
   60. The mutein, method, or composition of any of the preceding EEs which induces less pain relative to wildtype NGF.
   61. A method of treating a condition comprising delivering the NGF mutein of any one of EEs 1-15 to an anatomical region selected from ciliary body, sclera, retina, lens, vitreous body, optic nerve, choroid, cornea, iris, and anterior humor, optionally wherein the mutein is topically administration (e.g. the delivery results from topical administration of the NGF mutein to the eye).
   62. A method of treating a condition of an anatomical region comprising delivering the NGF mutein of any one of EEs 1-15 to the anatomical region, wherein the an anatomical region is selected from ciliary body, sclera, retina, lens, vitreous body, optic nerve, choroid, cornea, iris, and anterior humor optionally wherein the mutein is topically administered (e.g. the delivery results from topical administration of the NGF mutein to the eye).
   63. The method of EE 61 or 62, wherein the administration results in substantially increased half-life of the NGF mutein in the anatomical region relative to wild-type NGF.
   64. The method of EE 61 or 62, wherein the administration results in substantially increased concentration of the NGF mutein in the anatomical region relative to wild-type NGF.
   65. The method of EE 61 or 62, wherein the administration results in substantially increased concentration of the NGF mutein in the anatomical region relative to wild-type NGF at two hours following administration.
   66. The method of EE 61 or 62, wherein the administration results in substantially increased concentration of the NGF mutein in the anatomical region relative to wild-type NGF at six hours following administration.
   67. The method of EE 61 or 62, wherein the administration results in substantially increased concentration of the NGF mutein (e.g. at least five times concentration) in the anatomical region relative to wild-type NGF at 24 hours following administration.
   68. The method of any of EEs 63-67, wherein the anatomical region is an optical nerve or a retina.
   69. The NGF mutein of any one of EEs 1-15, wherein upon topical administration, the concentration of the NGF mutein in an anatomical region selected from ciliary body, sclera, retina, lens, vitreous body, optic nerve, choroid, cornea, iris, and anterior humor results in increased concentration relative to topical administration of wild-type NGF, and optionally wherein the topical administration is to the eye at 200 μg/ml, optionally wherein the administration is administration to the eye of a rabbit.
   70. The NGF mutein of any one of EEs 1-15, wherein topical administration results in increased half-life of the NGF mutein in an anatomical region selected from ciliary body, sclera, retina, lens, vitreous body, optic nerve, choroid, cornea, iris, and anterior humor when compared to topical administration of wild-type NGF (i.e. compared to the half-life of wild-type NGF in the anatomical region following administration of wild-type NGF) and optionally wherein the topical administration is topical administration to the eye of a rabbit at 200 μg/ml.
   71. The NGF mutein of EE 69 or 70, wherein the administration results in substantially increased concentration of the NGF mutein in the anatomical region relative to wild-type NGF at two hours following administration (i.e. relative to the concentration of wild-type NGF in the anatomical region following administration of wild-type NGF).
   72. The NGF mutein of EE 69 or 70, wherein the administration results in substantially increased concentration of the NGF mutein in the anatomical region relative to wild-type NGF at six hours following administration.
   73. The NGF mutein of EE 69 or 70, wherein the administration results in substantially concentration of the NGF mutein (e.g. at least five times concentration) in the anatomical region relative to wild-type NGF at 24 hours following administration.
   74. The NGF mutein of any of EEs 69-73, wherein the anatomical region is an optical nerve or a retina.

### EXAMPLES

### Example 1. Expression and Purification of the Wildtype Mature NGF from Hek293 Cells with Comparable Ex Vivo Activity To Murine NGF.

Nucleic acid-encoding the wildtype preproprotein of NGF (SEQ ID NO. 1) was inserted into the expression plasmid pSecTag2c. HEK 293 cells were stably transformed using the linearized expression plasmid with mouse Ig kappa signal peptide sequence (FIG. 3) and CMV promoter

Transformants were adapted to serum-free suspension culture and continually split to larger flasks. A typical suspension culture was inoculated at 0.5×10⁶ cells per mL and in 5 to 6 days HEK 293 cells grew typically over 3.5×10⁶ cells per mL. The cells were split every 3-4 days and secreted proteins in conditioned media were collected.

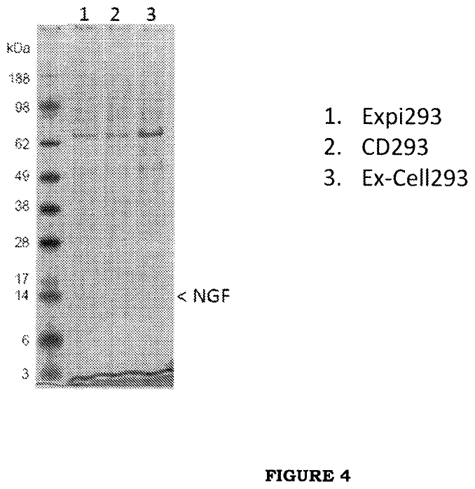

The expression and processing of pro-NGF was compared using various promoters, signal peptides, and commercial cell culture media. When the native signal peptide of NGF was used, no mature NGF was detectable. When the promoter of Ig Kappa was used, mature NGF was still poorly generated but detectable with Western blot analysis. Pro-NGF was efficiently processed into mature NGF only in Gibco Expi293™ expression medium (Thermo Fisher Scientific), while pro-NGF was expressed and cleaved in Ex-Cell® 293 serum free medium to a lower level (Millipore Sigma) or CD293 medium (Thermo Fisher Scientific) (FIG. 4). Mature NGF was produced at a low level when the cells were switched from Expi293™ medium to CD293 or Ex-Cell® 293 serum free medium. In contrast, switching from CD293 or Ex-Cell® 293 serum free medium to Expi293™ medium restored efficient production of mature NGF.

The production was routinely scaled up to 1L spinner. Culture medium was treated with 1% Triton X-100 for 30 minutes at room temperature as a virus inactivation process. The supernatant was loaded on an affinity column (IMAC) column and NGF eluted with a salt gradient. After diluting the salt concentration, NGF fraction was loaded on a strong cation exchange (SP) column and NGF eluted with a salt gradient.

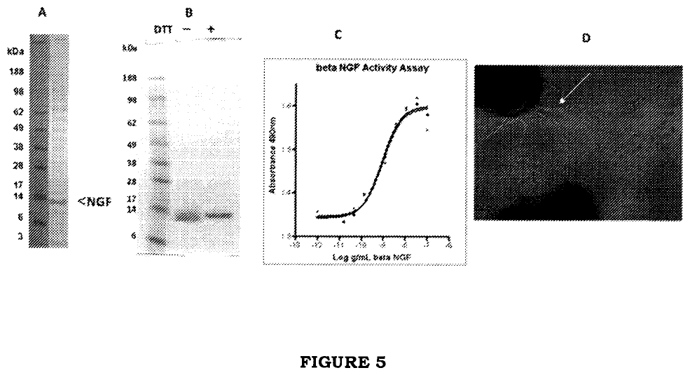

The proteins from clarified supernatants of the conditioned media as described above were separated on SDS polyacrylamide gel electrophoresis. The analysis shows pro-NGF was properly processed into mature NGF. The protein is composed of two non-covalent monomers, each with an apparent molecular mass of 13 kDa (FIG. 5). This mass corresponds to the mass of the known sequence of mature wild type human NGF (SEQ ID NO: 2). The purity is >98% with endotoxin at ≤1.5 EU/mg. Human TF1 erythroleukemic cells express TrkA receptor only. This cell-based proliferation assay was used for routine assay of NGF biological activity. Human NGF induced the cell proliferation with the potency comparable to that of the native murine NGF. NGF also induces the outgrowth of neurite. Production of NGF was scaled up to 10 L in a 50 L wave bioreactor which maintained high cell density, high viability, and high expression yield.

### Example 2. Increased Biological Activity of Instant NGF Muteins for TrkA

NGF mutations were made by making substitutions in amino acid residues, K34, R69, and R100 relative to SEQ ID NO: 2, and tested for effect against p75^{NTR}.

Twelve substitutions were introduced and compared for protein expression level and neuron survival activity: K34A, K34D, K34E, R69A, R69E, R100D, R100E, K34A/R69A, K34D/R69A, K34D/R100E, K34A/R69A/R100E, K34D/R69A/R100D. Mutated proteins were expressed in HEK293 cells using Expi293™ medium and purified to homogeneity. In order to assess the bioactivity of NGF on human receptor TrkA, the wildtype and its variants were tested using TF1 erythroleukemic cell-based proliferation assay. TF1 erythroleukemic cells express human TrkA and not p75^{NTR}.

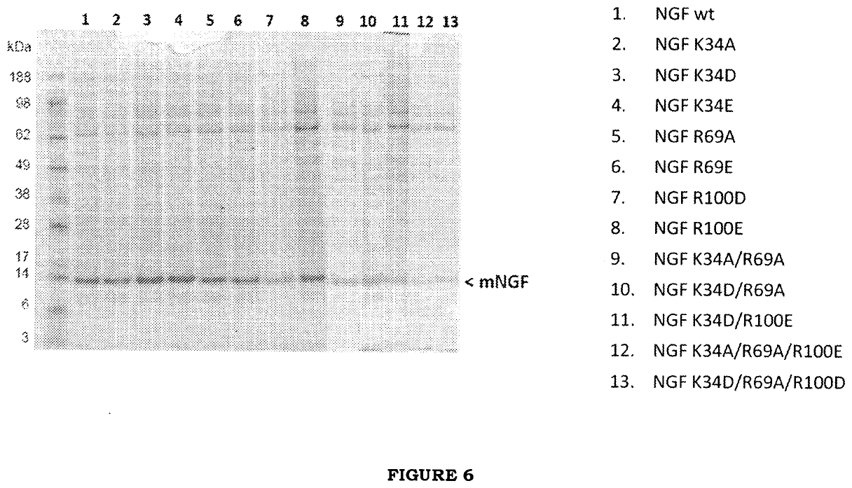

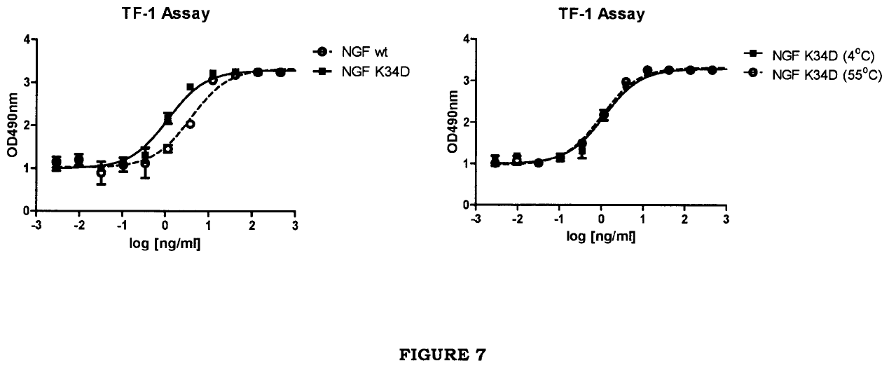

NGF and variants including instant muteins were pre-incubated at 4° C. and 55° C. for 7 days. Cells were then exposed to NGF and variants (0.1-200 ng/ml) in RPMI-1640 containing 10% FBS. Control wells were included, containing TF1 cells in the absence of NGF. Each treatment was performed in triplicate. After 24 h incubation period at 37° C., 5% CO₂, the absorbance at 490 nm was recorded using an ELISA reader. The expression levels of three single mutants (K34D, K34E, R100E) were comparable or higher compared to the wildtype. In contrast, other single mutants (K34A, R69A, R69E, R100E) and all the double or triple mutants showed reduced protein expression (FIG. 6). Overall, seven variants were excluded due to much lower ex vivo activity compared to the wildtype (Table 1). No significant effects of any mutation were anticipated on the growth of human TF1 erythroleukemic cells only expressing TrkA receptor, as no direct interaction was found or predicted with TrkA. Unexpectedly, the EC₅₀ for stimulating cell proliferation with K34D was reduced by more than 3 fold compared to the wildtype (FIG. 7), while the EC₅₀ for K34A was also reduced by 2 fold.

TABLE 1
EC₅₀ of the human wildtype NGF and its variants
in TF-1 proliferation assay

| | EC50 (ng/mL) | |
|---|---|---|
| Mutant | 4° C. | 55° C. |
| NGF wildtype | 3.70 | 3.10 |
| NGF K34A | 1.50 | 1.80 |
| NGF K34D | 1.08 | 0.92 |
| NGF K34E | 2.79 | 2.89 |
| NGF R69A | 105.5 | |
| NGF R69E | 53.9 | |
| NGF R100D | 6.84 | 17.13 |
| NGF R100E | 3.99 | 5.23 |
| NGF K34A/R69A | 9.13 | |
| NGF K34D/R69A | 12.26 | |
| NGF K34D/R100E | 58.80 | |
| NGF K34A/R69A/R100E | 689.3 | |
| NGF K34D/R69A/R100D | 23.02 | |

These results show that K34D and K34A have decreased EC₅₀ in TF-1 proliferation assay demonstrating enhanced TrkA bio-activity signaling (Table 1).

The in vitro thermal stability of the wildtype and five selected variants, K34A, K34D, K34E, R100E, R100D, were compared in triplicate for 7 days of storage in aliquots at 4 and 55° C. Loss of bioactivity was evaluated using TF1 cell proliferation assay. The three variants, K34A, K34D, K34E, retained or increased thermostability relative to wild type as demonstrated in Table 1. In contrast, the thermostability of R100D and R100E was reduced.

### Example 3. p75^{NTR} Stimulating Activity of Instant NGF Muteins.

To compare the bioactivity of the variants to the wildtype selectively through the neurotrophine receptor P75NTR, purified primary rat oligodendrocytes culture, which express P75^{NTR}, but not TrkA receptor (Malerba F et al. PLoS ONE, 2015), are used. Purified cultured rat OPCs primary cultures are prepared from brain cortices of postnatal day 1 Wistar rats by mechanical dissociation Bipolar NG2+ cells are immature OPCs which will undergo differentiation to become multipolar, post-mitotic pre-oligodendrocytes and acquire O4+ immunoreactivity. After 3 days in DM, cell medium is added with the wildtype or variants, followed by immunochemistry using NG2 and O4 monoclonal antibodies. Cell viability is assessed through the 3-(4,5-dimethyl thiazol -2-yl)-2,5-diphenyl tetrazolium bromide. Microplates are read on microplate reader, using wavelength of 550 nm. The wildtype inhibits differentiation and increases the percentage of the undifferentiated NG2+ cells while reducing the percentage of the differentiated O4+ cells in comparison to the untreated control group. The variants are less effective than the wildtype. Particularly, K34D is even less effective than K34A or K34E. The data are consistent with the reduced binding to p75^{NTR}.

### Example 4. Neuroprotective Effect of Instant NGF Muteins in a 112 Day Glaucoma Model of Rat.

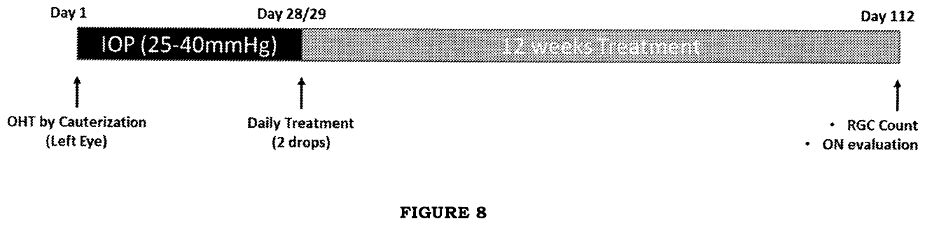

The purpose of the study was to test the efficacy of instant NGF muteins to preserve the retinal ganglion cell (RGC) in an experimental Ocular Hypertension (OHT) model of glaucoma by episcleral vein cauterization in Brown Norway rats over 16 weeks (FIG. 8). Briefly, the episcleral veins in one eye were cauterized while contralateral eye served as a sham. IOP was measured using a Tonopen XL applanation tonometer immediately after episcleral vein cauterization surgery and every week until the end point of each experiment. In the glaucoma model, ˜1.7-fold elevated IOP could be maintained for as long as 4 months. 61 animals were assigned to the study. A total of 57 Brown Norway rats were divided into five groups (n=10-12/group). Six (6) animals out of 61 animals were removed from the study because the IOP was not elevated or they had corneal abnormalities, and only 57 animals were administered with vehicle control and test articles on Day 29 (FIG. 8).

The day of the cauterization procedure was designated as Day 0. Clinical observations along with simultaneous moribundity/mortality checks were conducted and animals were weighed prior to the cauterization procedure. Ophthalmic examination and intraocular pressure (IOP) measurements were obtained prior to the cauterization procedure. The episcleral veins in one eye per animal in all the groups were cauterized on Day 0. The IOP was elevated by obstructing the aqueous humor outflow pathway more distally at the episcleral veins. The cauterized eye was called the OHT eye and the contralateral eye was called the Control eye. The OHT eyes in all groups was instilled with 10 μL of test or control article two times per day from Day 29 to Day 32, 8 or 10 μL on day 33, and 8 μL from Day 34 to Day 112 . Clinical observations along with simultaneous moribundity/mortality checks were performed daily until sacrifice. Body weights, ophthalmic examinations and IOP measurements were obtained weekly after the cauterization procedure, and prior to sacrifice. Electroretinograms (ERG) were recorded in both eyes for all animals per group at Week 12. Animals were sacrificed humanely at Week 16. Half of the OHT eyes per group were enucleated and fixed in 4% paraformaldehyde (PFA) for RGC Immunohistochemistry (IHC). The other half of animals per group were enucleated for collection of the optical nerves and the retinas for enzyme-linked immunosorbent assay (ELISA) analysis. Intraocular pressure (IOP) was elevated by EVC prior to the treatments from 20 to 28 mmHg. After 12 weeks, no treatments affected IOP.

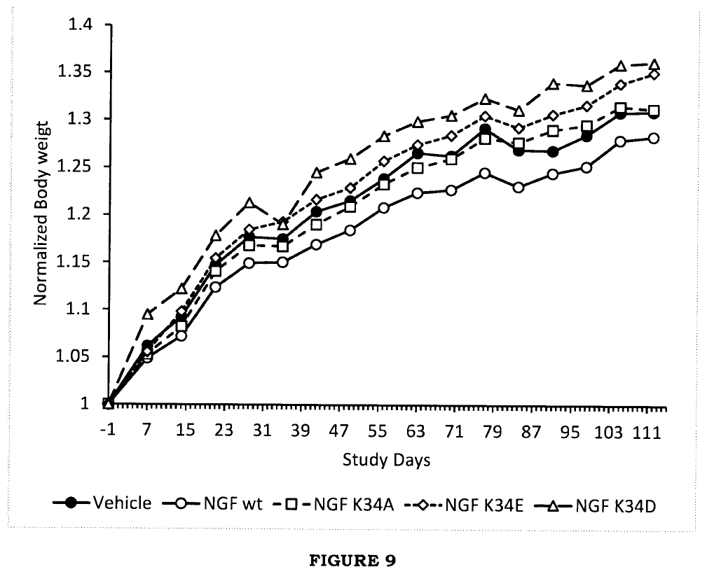

NGF K34D led to improved body weight compared to the placebo, indicating the animals were healthier (FIG. 9).

Both eyes from the surviving animals in the anterior and posterior segments were evaluated during the course of the study. The right eyes (Control eyes) in all animals were normal during the course of the study. The left eyes (OHT eyes) in most of the animals were normal except some animals with conjunctival redness with scores ranging from 1-2, conjunctival chemosis scored as 1, corneal opacity scores ranging from 1-2 and corneal opacity areas scored as a 1. These findings may be related corneal dry out due to multiple sedations, and are likely not related to the test articles.

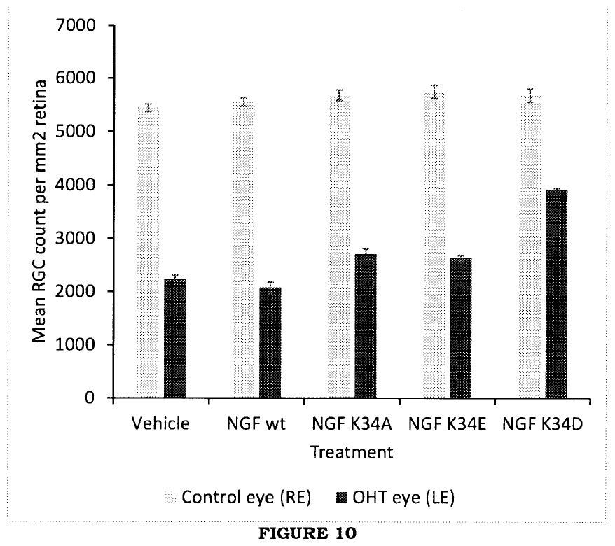

At the experimental end point, both eyes were enucleated and retinas were flat-mounted on a glass slide with the vitreous side up. Pictures for each retina were taken using a fluorescence microscope (Carl Zeiss Meditec, Jena, Germany), with 12 pictures/retina at ×20 magnification. Fluorogold retrograde labeling measured RGC retrograde transport. Because transport deficits preceded glaucomatous RGC death, quantification of labeled RGCs was a valid measure of RGC death. In all cases, manual RGC counting was performed by two independent persons masked to the protocol. Results from the three counts were averaged. The RGCs from the total of 56 retinas from the 27 animals (6 from the vehicle group, 6 from the wildtype group, 5 from Group NGF K34A group, 5 from NGF K34E group, 5 from NGF K34D group) were analyzed. The mean loss of RGCs at Week 16 were 58.56±3.39% (Mean ±SD), 62.79±3.97%, 52.33±5.25%, 54.16 ±2.67%, 30.92 ±4.06% respectively (FIG. 10). The data indicate the treatment with the wildtype NGF at 200 μg/ml as eye drop was not effective, while NGFK34A and NGFK34E at 200 μg/ml marginally reduced the RGC loss by 12 and 9%, respectively, compared to the vehicle group. The EC₅₀ of K34D was 35% lower than the one for NGF K34A. Unexpectedly, NGF K34D at 200 μg/ml was far more effective, reducing RGC loss by 50%.

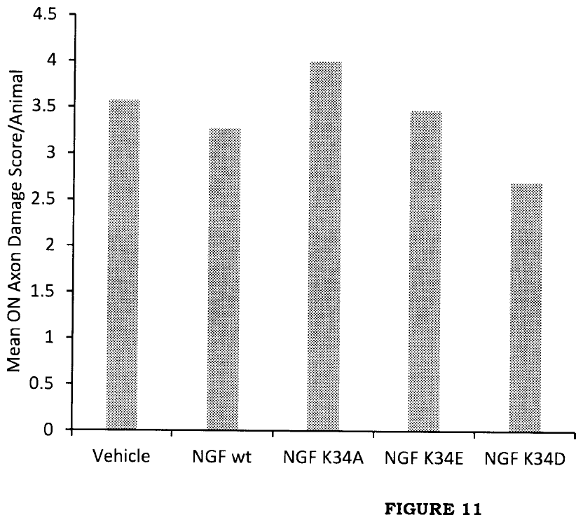

The optic nerve axon samples from 33 animals were prepared for plastic embedding, staining, and sectioning processing, but only 27 animals' samples were stained (6 from the vehicle group, 3 from the wildtype group, 4 from NGF K34A group, 3 from K34E group, and 6 from Group K34D group) (FIG. 11) and analyzed. The data from total scores divided by number of animals per group showed that the scores of NGF K34D were lower than those in the other groups. The results demonstrated that treatment with NGF K34D at 200 μg/mL effectively reduced optic nerve axondamage induced by the elevated IOP.

In summary, NGF K34D at 200 μg/mL effectively protected the structure and survival of RGCs and axons while the wildtype NGF and NGF K34A or K34E at 200 μg/mL were not effective or only marginally effective.

### Example 5: Pain Sensitivity Induced by the Wildtype NGF and NGF K34D In Rats

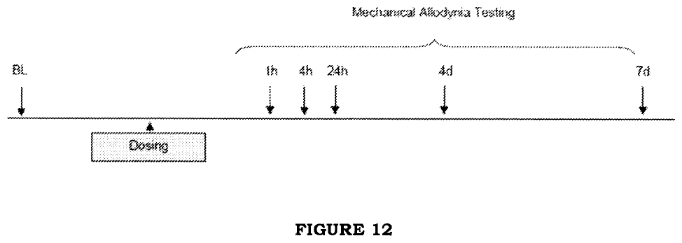

Mechanical allodynia was induced by intraplantar injections of either the wildtype NGF or the mutant NGF K34D. Mechanical allodynia was assessed using von Frey filaments to determine paw withdrawal thresholds prior to injection of 1, 3, or 10 μg of either NGF or NGF K34D within a volume of 50 μL normal saline subcutaneously into the plantar surface of male Sprague-Dawley rats. Another group of ten animals received 50 μL of normal saline. 70 total animals were thus split into seven groups of ten (FIG. 12). Prior to injections, pre-dose paw withdrawal thresholds were measured. Intraplantar injections were administered at time=0 and behavioral data was collected at 1 hr, 4 hr, 24 hr, 4 days and 7 days following the injection. No tissue or blood samples were collected.

Mean ipsilateral withdrawal thresholds of animals treated with either NGF WT or NGF K34D at all doses were significantly reduced at multiple time points following administration when compared to respective contralateral thresholds. Similarly, maintenance of reduced ipsilateral paw withdrawal thresholds following dosing was observed within both NGF WT- and NGF K34D-treated animals over time when compared to pre-dose thresholds.

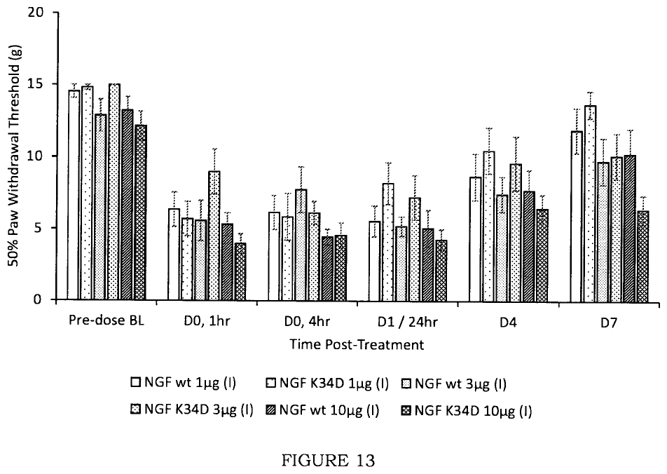

In addition, ipsilateral mechanical allodynia induced by administration of the same doses of NGF WT or NGF K34D did not significantly differ from each other within any time point post-dose (FIG. 13). In contrast, mean ipsilateral withdrawal thresholds of animals treated with normal saline did not differ at any time point following administration when compared to contralateral thresholds. Measured by the ratio of EC₅₀ for pain vs EC₅₀ for RGC-protective efficacy in the glaucoma model, NGF K34D exhibits significantly improved therapeutic index compared to the wildtype NGF, indicating that the instant NGF muteins can provide effective therapy for humans.

### Example 6. Eliminating the Binding to p75NTR while Retaining TrkA Biological Activity of Instant NGF Muteins.

NGF muteins were generated by making substitutions in amino acid residues, K34D, K32A, K32D, K34D/K32A, and K34D/K32D.

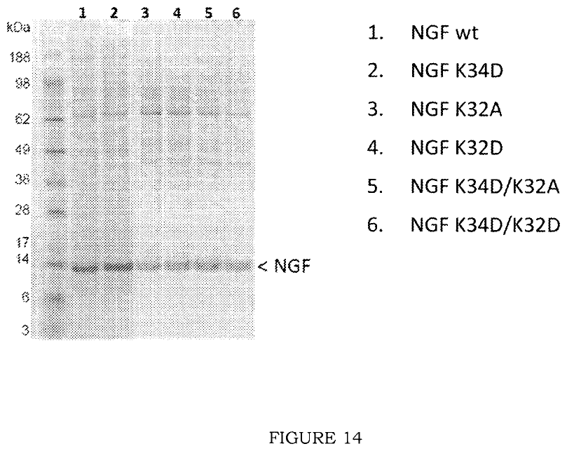

Mutated proteins were expressed in HEK293 cells using Expi293™ medium and purified to homogeneity. The expression levels of these muteins were comparable to the wildtype (FIG. 14).

The bioactivities of the wildtype NGF and its variants on human receptor TrkA were tested using TF1 erythroleukemic cell-based proliferation assay, as described in Example 2. The EC₅₀ for stimulating cell proliferation with K34D or K32A was reduced by about half compared to the wildtype (Table 2). The EC₅₀ for K32D was increased by 2 fold, and the EC₅₀ for both K34D/K32A and K34D/K32D was comparable to the wildtype NGF. Comparison of the activity after one week storage at 55 vs 4° C. indicates that the K34D/K32A mutant was less stable and the EC₅₀ was increased by 5-fold after the storage at 55° C. In contrast, NGF34D/32 D and K34D and K32D were remarkable stable at 55° C. compared to at 4°.

TABLE 2
EC₅₀ (ng/ml) and thermostability of the human wildtype NGF and its
variants in TF-1 proliferation assay.

| | 4° C. | 55° C. |
|---|---|---|
| NGF wt | 2.02 | |
| NGF K34D | 1.01 | 1.55 |
| NGF K32A | 1.26 | 1.12 |
| NGFK32D | 4.79 | 5.91 |
| NGFK34D/K32A | 1.91 | 9.7 |
| NGF34D/K32D | 2.62 | 1.15 |

The binding experiments were performed on Biacore 3000 at 25° C. Receptors were directly immobilized by direct immobilization using EDC/NHS amine coupling kit and GE protocol. The receptor was coated until the desired RU. The unoccupied active sites on the chip were quenching by flowing 1 M ethanol amine. The wildtype NGF or muteins flowed over the surface to measure binding. The receptor free flow cell 1 used as reference surface to account for any nonspecific binding of the wildtype NGF or its muteins to the surface. Data were analyzed using Biacore 3000 bia evaluation software (Biacore) using the values after reference subtraction. KD was determined by applying either local (for scouting) or global (full kinetics) 1:1 Langmuir fitting model. Full kinetics was performed with the range of analyte at a 2-fold serial dilution and flowed over the ligand from lowest to highest concentration range.

TABLE 3
Binding kinetic analysis of NGF and its variants to the TrkA and P75^{NTR} receptors. Both receptors were
immobilized on the chip, the concentrations of NGF and its variants were by 1:1 serial dilutions.

| Analyte | Trk (kinetic) | TrkA (equilibrium) | p75^{NTR} (kinetic) | p75^{NTR} (equilibrium) |
|---|---|---|---|---|
| NGF wt | kₐ = 2.29 × 10⁷ M⁻¹ · s⁻¹ | K_{D} = 3.44 × 10⁻¹¹ M | kₐ = 3.72 × 10⁷ M⁻¹ · s⁻¹ | K_{D} = 3.19 × 10⁻¹⁰ M |
| | k_{d} = 7.88 × 10⁻⁴ s⁻¹ | | k_{d} = 0.0119 s⁻¹ | |
| NGF K34D | kₐ = 6.87 × 10⁶ M⁻¹ · s⁻¹ | K_{D} = 1.22 × 10⁻¹⁰ M | kₐ = 2.73 × 10⁶ M⁻¹ · s⁻¹ | K_{D} = 8.62 × 10⁻⁹ M |
| | k_{d} = 8.34 × 10⁻⁴ s⁻¹ | | k_{d} = 0.0235 s⁻¹ | |
| NGF | kₐ = 6.54 × 10⁶ M⁻¹ · s⁻¹ | K_{D} = 6.07 × 10⁻¹⁰ M | kₐ = NA | K_{D} > 200 × 10⁻⁹ M |
| K34D/K32D | k_{d} = 3.98 × 10⁻³ s⁻¹ | | k_{d} = NA | |

TABLE 4
Receptor binding selectivity.

| | TrkA receptor | | | | P75^{NTR} | | |
|---|---|---|---|---|---|---|---|
| | Bio-activity | % of the | Binding | % of | Binding | % of the | Binding selectivity |
| | (ng/ml) | wildtype | affinity (nM) | wildtype | affinity (nM) | wildtype | (TrkA/p75^{NTR}) |
| NGF wt | 2.02 | | 0.034 | | 0.32 | | 9.4 |
| NGF K34D | 1.01 | 200 | 0.12 | 28 | 8.62 | 0.037 | 72 |
| | | | | (3.5 fold) | | (27 fold) | |
| NGF34D/K32D | 2.62 | 77 | 0.61 | 5.9 | >200 | <0.0016 | >328 |
| | | | | (18 fold) | | (625 fold) | |

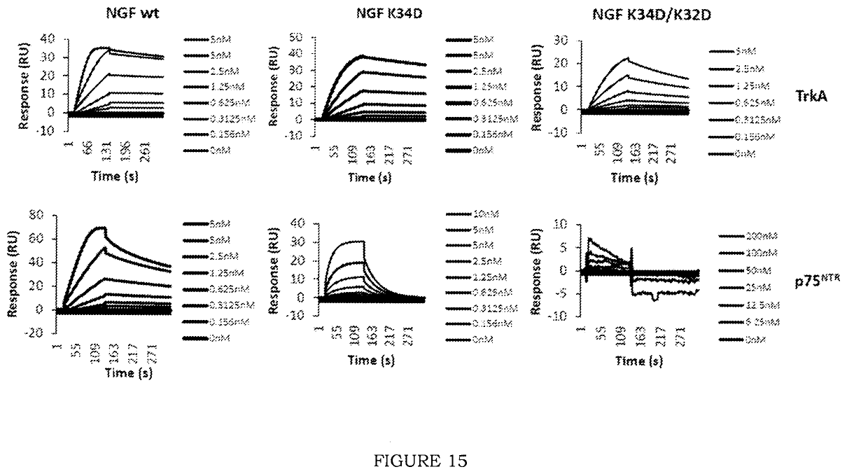

Based on the full kinetics data (FIG. 15), the binding affinity to TrkA was reduced by 3.5 and 18-fold for NGF K32D and NGF K34D/K32D respectively compared to the wildtype NGF (Table 3) but surprisingly with comparable or better biological effect (Table 2). Strikingly, the binding affinity towards p75^{NTR} was reduced by 27-fold with NGF K34D and the binding was no longer detectable with NGF K34D/K32D (Table 3). Together, these data indicate that NGF wt favors the binding to TrkA over p75^{NTR} by approximately 9-fold. NGF K32D is more TrkA-selective, favoring the binding to TrkA by 72-fold and NGF K34D/K32D is even more TrkA-specific ,with undetectable binding to p75^{NTR}. The double mutein favors the binding to TrkA by >328-fold (Table 4),

### Example 7 Neuroprotective Effect of Instant NGF Muteins in a 140 Day Glaucoma Model of Rat.

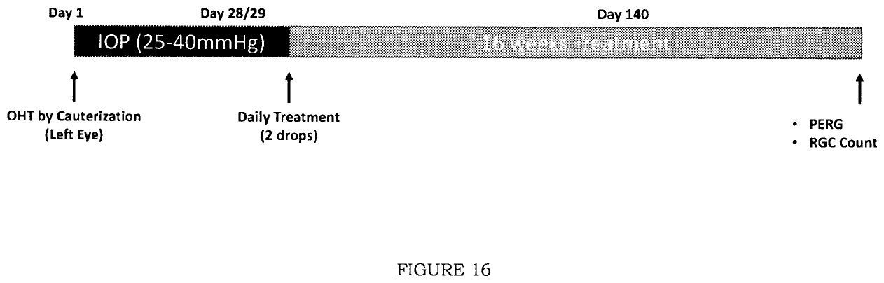

The purpose of the study was to test the efficacy of instant NGF muteins to preserve the retinal ganglion cell (RGC) in an experimental Ocular Hypertension (OHT) model of glaucoma by episcleral vein cauterization in Brown Norway rats over 20 weeks (FIG. 16), similar to the experiment described in Example 4.

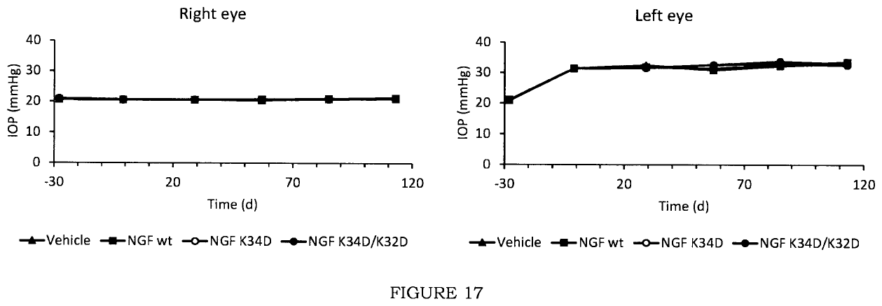

Body weights, ophthalmic examinations and IOP measurements were obtained weekly after the cauterization procedure, and prior to sacrifice. Pattern electroretinograms (PERG) were recorded in the OHT eyes for all animals at Week 20. Animals were sacrificed humanely at Week 20. Both eyes were enucleated and fixed in 4% paraformaldehyde (PFA) for RGC Immunohistochemistry (IHC). Intraocular pressure (IOP) was elevated by EVC prior to the treatments from 20 to 32 mmHg (FIG. 17). The treatments were initiated on day 28. No treatments affected IOP.

Both NGF K34D and NGF K34D/K32D led to improved body weight compared to the placebo, indicating the animals were healthier.

The pattern electroretinogram is the most specific technique for electrophysiological assessment of RGC function in the rodent models of glaucoma, which is dominated by inner retina activity whereas other retina activity is minimized. Dark-adapted pERG was performed on the OHT eyes of all animals before euthanasia. The animals were dark adapted for at least 8-12 hours before the pERG recording. The animals were anesthetized and placed on a table with heating pads. All animals had their pupils dilated with 1% tropicamide ophthalmic solution prior to pERG recording. The active electrodes made of gold wire were placed on the corneas. The reference electrodes were placed near the outer canthi, close to the eyes and the ground reference electrode was inserted in the back, subcutaneously, pERG was recorded to obtain at least the wave of Maximal Response (MR) from among the waves of the Rod Response (RR), MR and Oscillatory potentials (OP) in Dark-adapted pERG. The pERG Protocols were designed to meet the standards prescribed by the International Society for Clinical Electrophysiology of Vision (ISCEV) by Diagnosys LLC. After the pERG recording, an ophthalmic solution or ointment with or without antibiotic and/or balanced salt solution (BSS) was applied topically on both eyes to avoid corneas drying out, until the animals woke up and were returned to the cages.

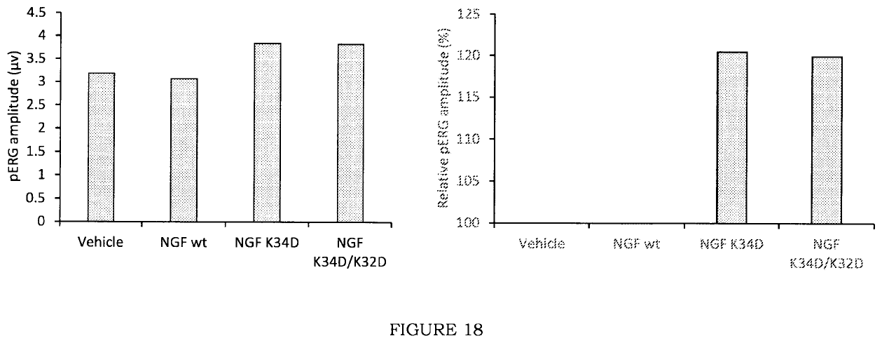

Eight animals per group had pERG with luminance 100 cd/m² at Week 20. Surprisingly, despite the EC50 comparable to the wildtype in the TF1 assay, the amplitude of the animals treated with K34D/K32D was 20% higher than the placebo control, which is similar to the one observed for K34D. In contrast, the wildtype NGF was not effective (FIG. 18).

Both eyes from the surviving animals in the anterior and posterior segments were evaluated during the course of the study. The right eyes (Control eyes) in all animals were normal during the course of the study. The left eyes (OHT eyes) in most of the animals were normal except some animals with conjunctival redness with scores ranging from 1-2, conjunctival chemosis scored as 1, corneal opacity scores ranging from 1-2 and corneal opacity areas scored as a 1. These findings may be related corneal dry out due to multiple sedations, and are likely not related to the test articles.

At the experimental end point, both eyes were enucleated and retinas were flat-mounted on a glass slide with the vitreous side up. Pictures for each retina were taken using a fluorescence microscope (Carl Zeiss Meditec, Jena, Germany), with 12 pictures/retina at ×20 magnification. The RGCs from the total of 32 retinas from the 32 animals. The data indicate that both NGFK34D and NGFK34D/K32D as an eye drop at 200 μg/ml is therapeutically effective and dramatically prevents RGC death . In contrast, the treatment with the wildtype NGF at 200 μg/ml was not effective and showed no difference compared to the vehicle control, consistent with the general failure of wildtype NGF in the art to treat glaucoma and prevent TGC death.

In summary, both NGF K34D and NGF K34D/K32D at 200 μg/mL twice daily effectively protected both survival and function of RGCs while the wildtype NGF at 200 μg/mL was not effective.

### Example 8. Pain Sensitivity Induced by the Wildtype NGF and NGF K34D/K32D in Rats.

The study was described as Example 5. 70 out of 70 animals survived the study and were in overall good health over the course of the study. Mean ipsilateral 50% response thresholds of animals treated with NGF wt were significantly reduced at all time points following administration of 1, 3, and 10 μg of NGF when compared to contralateral 50% response thresholds via Mann Whitney U test. Similarly, maintenance of mechanical allodynia was observed via significant main effects of time within the Friedman test at each dose level throughout the pharmacological testing period. Also, through multiple comparison testing, 50% paw withdrawal thresholds at multiple time points and dose levels throughout the pharmacological testing period were significantly reduced when compared to the pre-dose baseline.

When comparing ipsilateral 50% response thresholds of vehicle-treated animals to that of NGF-treated animals, NGFwt-treated animals were found to have significantly reduced 50% response thresholds at all doses at 1, 4, and 24 hrs following administration, as well as at 3 μg and 10 μg at 7 days following administration. Animals treated with NGF K34D/K32D were observed to have overall fewer significant differences when comparing ipsilateral 50% response thresholds to vehicle-treated animals, with significantly reduced 50% response thresholds at 1 μg 1, 4, and 24 hrs following administration, and 10 μg 1 hr following administration.

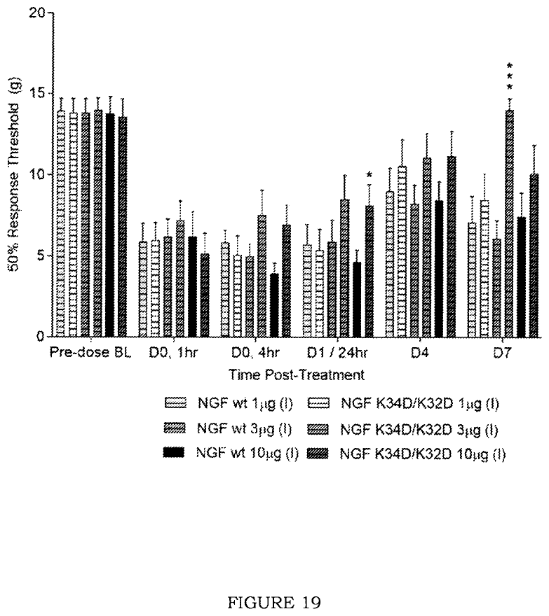

These data collectively suggest NGF wt induces persistent mechanical allodynia throughout the 7 day period while the allodynia effect of NGF K34D/K32D is significantly less persistent and becomes non-significant on days 4 and 7 (FIG. 19).

In conclusion, our data indicate both NGF K34D and NGF K34D/K32D muteins provide strong in vivo neuroprotection while NGF wt is not effective. Moreover, NGF K34D/K32D demonstrated a superior pain profile relative to NGF K34D by significantly reducing the side effect of pain, which significantly improve the efficacy by enabling higher dosage and better patient compliances. This improvement is particularly meaningful for treatment of chronic diseases with instant NGF muteins for which persist pain would otherwise make its clinical use intolerable.

The publications cited herein are incorporated by reference with respect to the referenced subject matter.

We claim:

1. An isolated human nerve growth factor (NGF) mutein comprising an acidic amino acid residue substitution at position 34 relative to SEQ ID NO. 2 and at least 80% sequence identity to SEQ ID NO. 2 or a fragment thereof that stimulates TrkA bio-activity.

2. The NGF mutein of claim 1 wherein the acidic amino acid residue substitution is aspartic acid, glutamic acid, N-Z-L-aspartic anhydride, 4-tert-butyl hydrogen 2-azidosuccinate, or γ-carboxy-DL-glutamic acid.

3. The NGF mutein of claim 1 wherein the acidic amino acid residue substitution is an aspartic acid.

4. The NGF mutein of claim 1 wherein the acidic amino acid residue substitution is a glutamic acid.

5. The NGF mutein of claim 1 further comprising a substitution at position 32 with an acidic amino acid.

6. The NGF mutein of claim 5, wherein the substitution at position 32 is an aspartic acid, glutamic acid, N-Z-L-aspartic anhydride, 4-tert-butyl hydrogen 2-azidosuccinate, or γ-carboxy-DL-glutamic acid.

7. The NGF mutein of claim 1 having the structure of FIG. 2 or an amino acid sequence differing from the structure of FIG. 2 by 5 substitutions or less among the amino acids other than X 1-X15 and Z, wherein each of X1-X12 is any natural or unnatural amino acid residue and wherein one or more of the Z are any acidic amino acid residue, and wherein each of X13-X15 is any natural or unnatural amino acid residue or is absent.

8. The NGF mutein of claim 1 wherein said mutein has at least 30% of the biological activity towards TrkA when compared to molar equivalent amount of wild type NGF.

9. The NGF mutein of claim 8 wherein the increased biological activity towards TrkA can be demonstrated by an EC50 in a TF1 erythroleukemic cell-based proliferation assay of not more than 170% of the EC50 of wild type NGF.

10. The NGF mutein of claim 1, wherein when expressed in a eukaryotic cell expression system, expression levels are at least 30% of the expression levels of NGF WT in an otherwise identical expression system.

11. An isolated polynucleotide comprising a DNA sequence that encodes the NGF mutein of claim 1.

12. An expression vector comprising the polynucleotide of claim 11.

13. The expression vector of claim 12 further comprising a polynucleotide coding for a pro-sequence.

14. The expression vector of claim 12 further comprising a polynucleotide coding for a leader sequence.

15. An isolated host cell transformed with the expression vector of claim 12.

16. The host cell of claim 15 wherein the host cell is a eukaryotic host cell, optionally wherein the eukaryotic host cell is a mammalian cell, optionally wherein the mammalian cell is a human cell, optionally wherein the human cell is a human embryonic kidney cell, optionally wherein the human embryonic kidney cell is a HEK293 cell.

17. A pharmaceutical composition comprising the NGF mutein of claim 1 in combination with a pharmaceutically acceptable carrier.

18. A method of treating a subject in need thereof comprising administering to the subject an effective amount of the composition of claim 17.

19. The NGF mutein of claim 1 , wherein the mutein, relative to wildtype NGF,
a. has reduced biological activity towards p75^{NTR} that can demonstrated by at least a 50% reduction in ° differentiation in a primary rat oligodendrocyte assay;
b. has reduced binding activity towards p75^{NTR} as demonstrated by a 100% increase in Kd for binding to p75^{NTR} as determined by BioCore3000;
c. retains at least 30% TrkA biological activity as demonstrated by a human TF1 erythroleukemic cell-based proliferation assay; and
d. has at least a 2-fold increase in the ratio of TrkA binding affinity over p75^{NTR} binding affinity; and
e. optionally, the mutein further comprises a substitution at position 32.

20. The NGF mutein of claim 19 wherein the acidic amino acid residue substitution is an aspartic acid.


SEQUENCE LISTING (2 sequences)

SEQ ID NO: 1 - protein, 241 aa, unidentified
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKRSSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 2 - protein, 120 aa, unidentified
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA